(19) United States
(12) Patent Application Publication
(10) Pub. No.: US 2001/0033839 A1
(43) Pub. Date: Oct. 25, 2001

(54) Vaccine and immunotherapy for solid nonlymphoid tumor and related immune dysregulation

(76) Inventor: Emilio Barbera-Guillem, Powell, OH (US)

Correspondence Address: M. Bud Nelson, BioCrystal Ltd., 575 McCorkle Boulevard, Westerville, OH, 43082-8888, US

(21) Appl. No.: 09/835,759

(22) Filed: Apr. 16, 2001

Related U.S. Application Data
(63) Continuation-in-part of application No. 09/411,116, filed on Oct. 4, 1999.

(51) Int. Cl. A61K 39/395; A61K 39/00

(52) U.S. Cl. 424/130.1; 424/184.1

(57) ABSTRACT

Provided are vaccines, methods of making the vaccines and methods for administering the vaccines for immunotherapy of an individual bearing, or at risk for developing, solid nonlymphoid tumor. The vaccine comprises an immunotherapeutic composition and tumor-associated antigen, and may further comprise one or more of an immunomodulator or a pharmaceutically acceptable carrier. A method of immunotherapy of an individual comprises administering to the individual an amount of the vaccine effective to suppress a TH2 response, and to induce a TH1 response against solid nonlymphoid tumor, in an individual having a TH2/TH1 imbalance.

This application is a continuation-in-part of co-pending application Ser. No. 09/411,116, which is herein incorporated by reference.

### BACKGROUND OF INVENTION

This invention relates, in general, to tumor vaccines. More particularly, the present invention relates to immunotherapy of solid nonlymphoid tumor which comprises tumor-associated antigen and a composition for treating the immune dysregulation associated with solid nonlymphoid tumor.

1. Immune Responses

Various populations of lymphocytes are involved in the induction of an immune response in an individual. One population, CD4+ lymphocytes (TH cells), can be distinguished into subsets on the basis of their secretion of cytokines (cytokine production or profile). Primarily, there are two subsets of TH cells, TH1 cells and TH2 cells. TH1 cells, which secrete cytokines that include but are not limited to IL-2 and interferon-gamma (IFN-γ), are generally accepted as being integral for cell mediated immunity. TH2 cells, which secrete cytokines that include but are not limited to IL-4, IL-6, IL-10, and macrophage-derived chemokine, support a humoral immune response. Similarly, a profile of cytokines can be produced by mononuclear cells, such as peripheral blood mononuclear cells, during immune stimulation. Thus, a TH1 immune response (“TH1 response”) can be distinguished from a TH2 immune response (“TH2 response”) based on detection of the cytokines produced; e.g., an immune response resulting in induction of cytokines comprising IL-12 and IFN-γ comprises a TH1 response, and an immune response resulting in induction of cytokines comprising IL-4, IL-10 and IL-6 comprises a TH2 response. The pathway of differentiation from naive precursor CD4+ T cells to either TH1 cells or TH2 cells, and the pathway of induction of a TH1 response or a TH2 response or a combination thereof, is determined by factors which include, but are not limited to, type of antigenic stimulation, the nature of the antigen, and influence of cytokines present during antigen presentation. Secreted cytokines characteristic of a TH1 response include IL-12 and IFN-γ. IL-12 acts as a differentiation factor on CD4+ T cells in promoting their specialization into IFN-γ-producing TH1 cells; promotes TH1 responses; inhibits neovascularization (angiogenesis); modulates migration and positioning of immune effector cells; and induces CD8+ cells to differentiate into cytotoxic CD8+ cells secreting a TH1 pattern of cytokines. IFN-γ acts on CD4+ T cells in promoting their differentiation into TH1 cells, and inhibits the proliferation of TH2 cells; increases class I MHC expression; and stimulates cytolytic activity of NK cells. Secreted cytokines characteristic of a TH2 response include IL-10, IL-6, IL-4, and macrophage-derived chemokine. IL-6 serves as a growth factor for activated B cells and plasma cells. IL-10 inhibits cytotoxicity of CD8+ cells; suppresses TH1 pattern of cytokine production by TH1 cells and other mononuclear cells; and inhibits T-cell mediated immunity which impairs effector T cell killing of emerging tumor cells which facilitates tumor progression. IL-4 promotes development of TH2 cells and secretion by mononuclear cells of a TH2 pattern of cytokines; stimulates expression of adhesion molecules on endothelial cells resulting in immobilization of immune effector cells to a site of inflammation; and inhibits development of cytotoxic CD8+ cells. Macrophage-derived chemokine is a potent chemoattractant for chronically activated TH2 cells.

2. Cancer

As known by those skilled in the art, the TH1 pattern of cytokine production and the TH2 pattern of cytokine production as detected in clinical samples from healthy individuals represents what is known in the art as a “TH1/TH2 balance”. In individuals (both animals and humans) bearing solid nonlymphoid tumors, tumor progression is associated with a change in the TH1/TH2 balance, in favor of a predominant TH2 response. For example, as compared to a TH1 /TH2 balance, in individuals bearing solid nonlymphoid tumor there has been demonstrated an increase in the number of TH2 cells as well as an induction in a TH2 response (known by those skilled in the art as a “TH2/TH1 imbalance”). Recent reports have demonstrated that tumor-infiltrating lymphocytes (TIL), and spleen lymphocytes from tumor-bearing individuals, preferentially produce a TH2 pattern of cytokines. Adenocarcinoma-associated effusions demonstrate a predominance of TH2 cytokines (e.g., IL-10) and decreased or undetectable amounts of TH1 cytokines (e.g., IL-12) consistent with a TH2/TH1 imbalance. Analysis of serum cytokine levels from individuals with advanced cancer also demonstrate a cytokine pattern consistent with a TH2/TH1 imbalance. A TH2/TH1 imbalance is viewed as an indication of an impaired cell mediated immunity. While current belief is that both cell mediated immunity and humoral immunity are important for antitumor immunity, it is generally accepted that a dominant TH1 response is critically important for the generation of a tumor-specific cell mediated immune response in induction of antitumor immunity. The mechanisms which underlie the generation of a TH2/TH1 imbalance, and the suppression of a TH1 response, in individuals bearing solid nonlymphoid tumor are not clearly understood. Mechanisms that have been proposed include down regulation of MHC molecule expression by tumor cells, lack of costimulatory molecule expression by tumor cells, production of immunosuppressive factors by tumor cells, and the presence of so-called “suppressor cells” induced by cancer.

3. Immunotherapy

The development of vaccines to induce antitumor immunity is dependent on understanding tumor biology, immune responses induced by tumor-associated antigens, and the interaction between host immune cells and tumor. Numerous vaccines have been developed, and continue to be developed. Vaccines comprise tumor-associated antigen in a form comprising whole tumor cells, fractions of tumor cells, tumor cell extracts, an isolated antigen, and a combination of more than one antigen. Tumor-associated antigen has been used in various vaccine types for cancer immunotherapy. Such vaccines are well known in the art (see, e.g., CancerNet of the National Cancer Institute which lists ongoing clinical trials for cancer, including doses and regimens; and reviews by Monzavi-Karbassi & Kieber-Emmons, BioTechniques 30:170-189, 2001, and Herlyn and Birbent, 1999, Ann. Med. 31:66-78; the disclosures of which are herein incorporated by reference), as exemplified in Table 1.

Key: *components include, but are not limited to, gangliosides (e.g., GM-2, GD2), MART-1, gp100, p53, mutant p53, Ras peptide, mutated ras peptides, MAGE-12, CEA, E6 & E7 peptides, rV-B7.1, MUC1, Thompsen-Friedenreich antigen, HER-2/neu peptides, ESO-1, Tyrosinase peptides, PSA, mutated von Hippel-Lindau peptides, sTn, CO17-1A/GA733, heat shock protein, TRP-1, TRP-2, tumor-derived exosomes.

TABLE 1

| Vaccine | Adjuvant/carrier/Treatment | Tumor |
|---|---|---|
| Cellular: | BCG, Freund's, Detox, | renal, melanoma, |
| autologous, allogeneic, | Vaccinia virus, interferon, | lung, colon, |
| virus-modified, | dinitrophenyl-P, dendritic | colorectal, |
| cytokine-transduced, | cells | pancreatic, |
| or hapten-modified | | ovarian |
| tumor cells | | |
| Viral lysates and other | Vaccinia virus, Newcastle | colorectal, |
| lysates of tumor cells | disease virus, dendritic | melanoma, ovarian, |
| | cells, KLH | renal |
| Tumor cell extracts | Irradiated, Detox, | lung, melanoma |
| | dendritic cells | |
| Tumor RNA | dendritic cells | adenocarcinomas, |
| | | renal, prostate, |
| | | breast |
| Component: | Detox, QS21, SB AS-2, | melanoma, prostate, |
| purified from tumor, | ISA-51, human papilloma | colon, breast, |
| recombinant, peptide, | virus dendritic cells, | renal cell, lung, |
| synthetic, or multi- | fowlpox virus, vaccinia | cervical, ovarian, |
| antigen (polyvalent)* | virus, peripheral blood | pancreatic, |
| | mono-nuclear cells, KLH, | gastrointestinal |
| | BCG | |
| Tumor DNA, DNA- | BCG, cytokine (IL-12, or | GD2-positive |
| based (encoding | IFN-γ) | tumors, colon or |
| antigen or anti- | | rectum, lung, |
| idiotype mimic of | | cervical, gp-100-ex- |
| antigen) | | pressing mela- |
| | | noma, CEA- or |
| | | ErbB-2-expressing |
| | | tumor |

As exemplified in Table 1, vaccines incorporating tumor-associated antigen are typically administered with one or more additional components. Thus, combinations comprising a vaccine typically include tumor-associated antigen-pulsed dendritic cells with or without an adjuvant and/or with or without one or more immunomodulators, adjuvant with tumor-associated antigen, tumor-associated antigen without adjuvant, tumor-associated antigen with one or more immunomodulators, tumor-associated antigen with adjuvant and one or more immunomodulators, and combinations thereof. Despite the number of vaccines being implemented, it is clear that cancer vaccines have failed to fulfill their promise as effective anti-cancer therapy (Monzavi-Karbassi & Kieber-Emmons, 2001, supra). For example, IL-12 has been used in vaccines, in conjunction with tumor-associated antigen, to induce a switch from the TH2/TH1 imbalance to a predominant TH1 response. However, recent reports indicate that IL-12 with initial vaccination is insufficient to sustain a long-term TH1 response, that even with supplemental boosting with IL-12 after initial vaccination the induced TH1 response is only transient, and that repeated exposure to antigen and IL-12 is necessary to maintain a persistent TH1 response to antigen. Thus, a better understanding of tumor biology, as well as the interaction between tumor and host immune cells, is necessary for the advancements in immunotherapy of tumors and vaccine development.

Thus, a long felt need exists for vaccines which can be used in the treatment and/or prevention of solid nonlymphoid tumors, and for vaccines which can both induce a cell mediated immune response and suppress a TH2 response in overcoming the TH2/TH1 balance in individuals bearing solid nonlymphoid tumors. More specifically, immunotherapy of tumor should ideally control the inappropriate and predominant TH2 response, rather than merely inducing a TH1 response. The present invention satisfies this need and additionally provides related advantages.

### SUMMARY OF INVENTION

The present invention provides vaccines and methods of vaccinating an individual so as to suppress a TH2 response, and induce a cell mediated immune response to tumor-associated antigen, in an individual having a TH2/TH1 imbalance. This multi-faceted approach is more effective in inducing and sustaining a TH1 response to a vaccine antigen than the uni-dimensional approach currently used in immunotherapy of tumors. The vaccines and methods of vaccinating according to the present invention can be used to improve the efficacy of existing tumor-associated antigens or enable newly discovered tumor-associated antigens, and to suppress a pre-existing TH2 response and induce a cell mediated immune response. Such improved efficacy may provide additional benefits which may include, but are not limited to, safe administration of optimal amounts of tumor-associated antigen comprising a combination of tumor antigens, induction of a more protective antitumor immunity than that inducible by current vaccines, and maintenance of a longer lasting antitumor immunity than that inducible by current vaccines.

The present invention relates to a discovery that in individuals with solid nonlymphoid tumor there can exist a humoral immune response to shed tumor antigens, a pro-tumor immune response, which promotes tumor progression. Further, it has been discovered that a pro-tumor immune response favors a polarization to a TH2 response, in bringing about the TH2/TH1 imbalance demonstrable in individuals bearing solid nonlymphoid tumor. More particularly, a pro-tumor immune response comprises an alteration in the TH1 /TH2 balance in favoring a TH2 response, and suppressing development of a cell mediated immune response comprising a TH1 response, a cytotoxic CD8+ T cell response, or a combination thereof. This immune dysregulation results in an impaired cell-mediated immunity and an activated humoral immunity which promote tumor progression. Subsequent attempts to perturb the immune system with a vaccine for inducing a cell mediated immune response comprising a TH1 response in attempts to overcome the TH2/TH1 imbalance, without reducing the TH2 response caused by a pro-tumor immune response, may be insufficient or ineffective to induce development of an antitumor immune response for mediating tumor regression. Thus, a pro-tumor immune response's suppressive events (such as mediated by B cells, immune complexes, and activated immune effector cells) represent an important factor responsible for the reduced efficacy of current cancer vaccines; and, hence, represent an important target for immunotherapy of tumors. The above and other objects, features, and advantages of the present invention will be apparent in the following Detailed Description of the Invention when read in conjunction with the accompanying drawings.

### BRIEF DESCRIPTION OF DRAWINGS

FIG. 1 is a bar graph illustrating a cytokine release assay for IL-6 produced and secreted by macrophages alone or when incubated with one or more various other components.

FIG. 2 is a bar graph illustrating a cytokine release assay for IL-4 produced and secreted by macrophages alone or when incubated with one or more various other components.

FIG. 3 is a bar graph illustrating a cytokine release assay for IL-10 produced and secreted by macrophages alone or when incubated with one or more various other components.

FIG. 4 is a graph illustrating the effect of various treatments on in vivo tumor progression.

### DETAILED DESCRIPTION

Definitions

The term “depletion” is used herein in reference to B cells, and for purposes of the specification and claims, to mean one or more of: blocking of B cell function; functional inactivation of B cells; cytolysis of B cells; inhibiting the proliferation of B cells; inhibiting the differentiation of B cells to plasma cells; causing a B cell dysfunction which results in an immunotherapeutic benefit; inhibiting secretion of cytokines or other tumor-promoting soluble factor(s) by activated B cells; reduction in the number of B cells; inactivation of B cells which have been primed or activated by shed tumor antigen; blocking of one or more functions (e.g., cytokine production or antigen presentation or the like) of B cells which have been primed or activated by shed tumor antigen (“shed tumor antigen-specific B cells”); cytolysis of B cells which have been primed or activated by shed tumor antigen; and reduction in the number of B cells which have been primed or activated by shed tumor antigen. B cell depletion may be a result of one or more mechanisms including, but not limited to, clonal inactivation, apoptosis, antibody-dependent cellular cytotoxicity, complement-mediated cytotoxicity, and a signal pathway mediated inactivation, dysfunction, or cell death.

The term “immunotherapeutic composition” is used herein, for purposes of the specification and claims, to mean a composition (a) comprised of at least one affinity ligand which selectively (preferentially) binds to at least one determinant present on nonmalignant B cells, preferably mature B cells and/or memory B cells; and (b) whereupon contact and binding to such B cells, directly or indirectly results in (causes and/or enables) B cell depletion when added in an amount effective to cause the B cell depletion. B cell depletion may preferably comprise depletion of shed tumor antigen-specific B cells, particularly in sites that are foci of a pro-tumor immune response. Treatment with an amount of the immunotherapeutic composition in an effective amount to result in B cell depletion may result in a beneficial function. Such a beneficial function may include, but is not limited to, one or more of: inhibiting the proliferation of B cells in lymphoid tissues which are a foci of the pro-tumor immune response; inhibiting secretion of TH2 cytokines by shed tumor antigen-specific B cells or their progeny; reducing the relative number (e.g., causing or enabling cytolysis) of B cells which have been primed or activated by shed tumor antigen; and inhibiting secretion of anti-shed tumor antigen antibody by shed tumor antigen-specific B cells or their progeny in reducing the amount of immune complexes formed. As an illustrative, but non-limiting, example, an anti-CD20 mAb, or an anti-Lym-1 mAb, or an anti-CD19 mAb or an anti-CD22 mAb or an anti-CD21 mAb, may selectively bind to mature and/or memory B cells (via CD20, Lym-1, CD19, CD22, or CD21, respectively) and facilitate or result in B cell depletion when added in an effective amount to result in B cell depletion. In another example, a bi-specific antibody mAb, anti CD3-CD19 mAb, may bind to T cells (via CD3) and B cells (CD19) to mediate T cell-B cell interactions that may facilitate B cell depletion when added in an effective amount to result in B cell depletion. In yet another example, the affinity ligand comprises a peptide which binds CD21 expressed by B cells (in blocking binding of CD21 to other molecules such as complement bound to immune complexes). Such peptides are known in the art (e.g., a peptide from gp350/220 envelope glycoprotein of Epstein Barr virus; Henchoz-Lecoanet et al., 1996, Immunology, 88:35-39, which is herein incorporated by reference). In another embodiment the affinity ligand, which comprises the immunotherapeutic composition, may further comprise at least one anti-B cell agent. The “anti-B cell agent” comprises a cytolytic agent (e.g., the agent itself or a vector that is introduced into B cells and therein the vector encodes a cytolytic agent). The anti-B cell agent may be coupled to the affinity ligand using methods known in the art for coupling affinity ligands to other molecules (See, for example, conjugates as reviewed by Ghetie et al., 1994, Pharmacol. Ther. 63:209-34; U.S. Pat. No. 5,789,554, the disclosure of which is herein incorporated by reference). Often such methods utilize one of several available heterobifunctional reagents used for coupling or linking molecules. The affinity ligand serves to selectively bind the B cells, thereby bringing the anti-B cell agent in contact with or in functional proximity of B cells. A cytolytic agent is an agent that, by interacting directly with such B cells, causes B cell cytotoxicity. Such cytolytic agents may include, but are not limited to, a therapeutically effective amount of toxins; drugs; enzymes; cytokines; radionuclides; photodynamic agents; and molecules which induce apoptosis (e.g., Fas ligand; a Fas ligand expressing vector has been described in more detail in by the present inventor in Gene Therapy 8:209-214, 2001, the disclosure of which is herein incorporated by reference). Toxins may include a cytolytically effective amount of ricin A chain, mutant Pseudomonas exotoxins, diphtheria toxoid, streptonigrin, boamycin, saporin, gelonin, pokeweed antiviral protein, or the like. Drugs may include an effective amount of cytotoxic drug including, but not limited to, fludarabine, chlorambucil, daunorubicin, doxorubicin (e.g., in liposomes), cisplatin, bleomycin, melphalan, mitomycin-C, and methotrexate. A preferred cytotoxic drug may be used as an anti-B cell agent in the present invention to the exclusion of a cytotoxic drug other than the preferred cytotoxic drug. Due to the sensitivity of B cells to radiation, a radionuclide may include, but is not limited to, a radiometal such as yttrium which emits a high energy beta particle, and I¹²⁵ that emits Auger electrons, that may be absorbed by adjacent B cells. A photodynamic agent may include a cytolytically effective amount of a porphyrin or a porphryin derivative as known in the art. A preferred anti-B cell agent may be used in the present invention to the exclusion of an anti-B cell agent other than the preferred anti-B cell agent. In another preferred embodiment, the immunotherapeutic composition, for purposes of the specification and claims, may comprise a composition for suppressing a humoral immune response by depletion of complement component C3; i.e., it is known in the art that cobra venom factor can suppress a humoral immune response by the depletion of C3. One mechanism of B cell activation by immune complexes comprising shed tumor antigen and anti-shed tumor antigen antibody is believed to involve crosslinking of the B cell receptor and CD21 on the surface of B cells. For example, shed tumor antigen of an immune complex binds to the B cell receptor, and complement bound to the immune complex binds to the complement receptor CD21 of B cells, thereby causing receptor crosslinking and activation of B cells. Additionally, it is known in the art that immune complexes can be targeted to follicular dendritic cells for subsequent antigen presentation (e.g., complement of the immune complexes binds to CD21 expressed on follicular dendritic cells), and that cobra venom factor can inhibit the binding of immune complexes to follicular dendritic cells. Thus, cobra venom factor may deplete B cells by functionally inactivating B cells from activation by receptor crosslinking mediated by immune complexes, as well as interfering with antigen presentation by follicular dendritic cells. A preferred immunotherapeutic composition may be used in the present invention to the exclusion of an immunotherapeutic composition other than the preferred immunotherapeutic composition.

The term “immunomodulator” is used herein, for purposes of the specification and claims, to mean one or more compositions that, when administered to an individual in an effective amount, induces a cell mediated immune response comprising a TH1 response, and more preferably, may also induce a cytotoxic CD8+ T cell response. As known to those skilled in the art, a composition that may induce a cell mediated immune response comprising a TH1 response may include, but is not limited to, IL-12, IL-12 and melatonin, flavone acetic acid (flavonoid, 2-heteroaryl flavonoid derivatives, flavone-8-acetic acid), QS-21(a purified form of saponin, at a high dose) and monophosphoryl lipid A, N-acetylcysteine, SAF-1 (Syntex adjuvant formulation-1), AS101 (ammonium trichloro (dioxoethylene-O,O′) tellurate), lentinan (a fungal branched 1→3-(beta)-D-glycan), TraT protein (“ISCAR” or immunostimulatory carrier- an integral membrane protein of E. coli), Viscum album extract (commercially available extract of mistletoe), Z-100 (a lipid arabinomannan-containing extract of M. tuberculosis), OK-432 (Picibanil, inactivated and heat treated S. pyogenes Su strain), immunostimulatory DNA sequences, and the like. For example, IL-12 has been shown to be a potent inducer of naive CD4+ cells towards a cell mediated immune response comprising a TH1 response. IL-12 may be administered to a human individual as a cytokine in solution (e.g., rlL-12 in a dose ranging from about 10 ng/kg to about 300 ng/kg, twice weekly, subcutaneously or intratumoral), or in the form of dendritic cells or fibroblasts genetically engineered to express human IL-12 (see, e.g., Lotze et al., 1997, Cancer J. Sci. Am. 3/S (S109-S114), herein incorporated by reference). In another example, short bacterial immunostimulatory DNA sequences containing unmethylated CpG motifs have been shown to be able to stimulate a TH1 response (e.g., by inducing IL-12 production), and hence stimulate a cell-mediated immune response (Roman et al., 1997, Nat. Med. 3:849-854; Lipford et al., 1997, Eur. J. Immunol. 27: 3420-3426, herein incorporated by reference). An amount of an immunomodulator effective to induce a cell mediated immune response comprising a TH1 response will vary depending on such factors as the mode of administration, individual's age, weight, general medical condition, and immune status. For purposes of illustration, but not limitation, lentinan has been administered intravenously (e.g., 2 mg, 3 times per week), melatonin has been administered orally (e.g., 20 mg/day in the evening), Viscum album has been administered subcutaneously (e.g., 2-3 times per week, ranging from 0.1 to 30 mg), AS101 has been administered by intravenous drip (e.g., in a range of from about 3 mg/m² to about 12 mg/m²), and QS-21 has been administered subcutaneously (e.g., in a range of from about 100 μg to about 200 μg). A preferred immunomodulator may be used in the present invention to the exclusion of an immunomodulator other than the preferred immunomodulator.

The term “pharmaceutically acceptable carrier” is used herein, for purposes of the specification and claims, to mean a medium that facilitates administration of the vaccine according to the present invention to an individual. Typically, the medium is sterile. Suitable pharmaceutically acceptable carriers are well known to those skilled in the art to include, but are not limited to, buffered saline solutions, buffered carbohydrate solutions, citrate buffers, liposomes (Phillips et al., 1994, J. Immunother, Emphasis Tumor Immunol. 15:185-93), sterile water, and the like.

The term “determinant” with reference to B cells, is used herein, for purposes of the specification and claims, to mean a molecule which is preferentially expressed by B cells, and more preferably, by memory B cells and/or mature B cells, wherein the molecule is involved in and responsible for selective binding to an affinity ligand having binding specificity and avidity for the determinant. Cell-associated determinants may include, but are not limited to, molecules, receptors, components, or surface immunoglobulin, present on the surface of the cell membrane. “Preferentially expressed” is used herein to mean that the cell-associated determinant is expressed on a substantial number (e.g., in a range of approximately 30% to 100%) of the B cells to which is targeted the immunotherapeutic composition. In a preferred embodiment, the determinant is primarily expressed on B cells, with little or no expression of the determinant (as relative to the number of cells expressing the determinant or to the level of expression as compared to B cells) by other subpopulations of immune cells (with the exception of follicular dendritic cells; e.g., when the determinant comprises CD21) contained within the region to which the immunotherapeutic agent is intended to be targeted. In a preferred embodiment, the determinant is selected from the group consisting of CD19, CD20 (see, e.g., U.S. Pat. No.5,776,456, the disclosure of which is herein incorporated by reference), CD21, CD22 (see, e.g., LL2, U.S. Pat. Nos. 5,789,554, 6,183,744, 6,187,287, the disclosures of which are herein incorporated by reference; Erickson et al., 1996, Int. Immunol 8:1121-9), Lym-1 (see, e.g., U.S. Pat. No. 5,789,554, the disclosure of which is herein incorporated by reference), CDIM (see, e.g., U.S. Pat. No. 5,593,676, the disclosure of which is herein incorporated by reference), sig having binding specificity for shed tumor antigen, CD79a, CD79b, CDw78, CDw75, CD72, B cell receptor, and a combination thereof.

The term “lymphoid tissue” is used herein, for purposes of the specification and claims, to mean a tissue which contains localized areas (e.g., follicles) of antigen presenting cells (e.g., follicular or germinal center dendritic cells) and B lymphocytes, and in which can be induced an immune response involving B cells. An example of such localized areas comprises germinal centers. Such lymphoid tissues comprise tissues may include, but are not limited to, lymph nodes, milky patches in the mesenterium of the intestine, omentum, appendix, Peyer's patches, loose connective tissue (e.g., associated with vessels in the walls of the aorta), lymphatic vessels, submucosal spaces, subserosa spaces, peritoneal cavity, ligaments (e.g., gastro-hepatic ligament), and the like.

The term “B cells” is used herein, for purposes of the specification and claims, and in reference to the vaccine, the methods of vaccination, and to a pro-tumor immune response comprising treating B cells involved therein, to mean mammalian (and preferably human) nonmalignant B cells. As known to those skilled in the art, malignant B cells refers to cancer cells of B cell origin, such as B cell lymphomas, and B cell leukemias. Thus, the term “B cells”, as used herein in reference to the compositions and methods of the present invention, specifically excludes B cell lymphomas, B cell leukemias, and cancer cells of B cell origin. In regards to the present invention, nonmaligant B cells are inclusive of one or more subpopulations such as memory B cells, mature B cells, and other subpopulations (e.g., immature B cells, shed tumor antigen-specific B cells, and the like) as will be more apparent from the following embodiments.

The term “metastases” is used herein, for purposes of the specification and claims, to mean metastatic cells from a primary tumor wherein the primary tumor is a solid nonlymphoid tumor, as will be more apparent from the following embodiments.

The term “affinity ligand” is used herein, for purposes of the specification and claims, to mean a molecule which has binding specificity and avidity for a determinant associated with B cells that may be present in lymphoid tissues, and/or infiltrating solid, nonlymphoid tumors, and/or circulating in body fluids such as peripheral blood. In general, affinity ligands are known to those skilled in the art to include, but are not limited to, lectins (or fragments or derivatives thereof which retain specific binding activity), monoclonal antibodies (“mAb”, including chimeric or genetically modified monoclonal antibodies which may be preferable for administration to humans), peptides, and aptamers. The term “monoclonal antibody” is also used herein, for purposes of the specification and claims, to include immunoreactive fragments or immunoreactive derivatives (e.g., peptides) derived from a mAb molecule, which retain all or a portion of the binding function of the whole mAb molecule. Such immunoreactive fragments or immunoreactive derivatives are known to those skilled in the art to include F(ab′)₂, Fab′, Fab, Fv, scFV, Fd′, Fd, and the like. Methods for producing the various fragments from mAbs are well known in the art (see, e.g., Plückthum, 1992, Immunol. Rev. 130:152-188). For example, F(ab′)₂ can be produced by pepsin digestion of the monoclonal antibody, and Fab′ may be produced by reducing the disulfide bridges of F(ab′)₂ fragments. Fab fragments can be produced by papain digestion of the monoclonal antibody, whereas Fv can be prepared according to methods described in U.S. Pat. No. 4,642,334. Single chain derivatives can be produced as described in U.S. Pat. No. 4,946,778. The construction of chimeric antibodies is now a straightforward procedure (Adair, 1992, Immunological Reviews 130:5-40) in which the chimeric antibody is made by joining the murine variable region to a human constant region. Additionally, “humanized” antibodies may be made by joining the hypervariable regions of the murine monoclonal antibody to a constant region and portions of variable region (light chain and heavy chain) sequences of human immunoglobulins using one of several techniques known in the art (Adair, 1992, supra, Singer et al., 1993, J. Immunol. 150:2844-2857). Methods for making a chimeric non-human/human mAb in general, and a chimeric anti-CD20 mAb and chimeric anti-CD22 mAb in particular, are described in detail in U.S. Pat. Nos. 5,736,137 and 6,187,287, respectively. The chimeric anti-CD20 antibody described in U.S. Pat. No. 5,736,137 and the chimeric anti-CD22 antibody described in U.S. Pat. No. 6,187,287, each have been reported to be therapeutically active on its own; e.g., does not require coupling to a toxin or radioisotope to induce cytolysis of targeted B cells. Likewise, crosslinking of a B cell by an anti-CDIM mAb has been reported to induce a cellular response ultimately resulting in cell death (U.S. Pat. No. 5,593,676). In a preferred embodiment, affinity ligands may include, but are not limited to, a mAb having binding specificity for one of CD19, CD20, CD21, CD22, CDIM, or Lym-1. Aptamers can be made against B cell determinants using methods described in U.S. Pat. No. 5,789,157 (the disclosure of which is herein incorporated by reference).

The term “solid non-lymphoid tumor” is used herein, for purposes of the specification and claims, to mean any primary tumor of ductal epithelial cell origin, including, but not limited to, tumors originating in the liver, lung, brain, lymph node, bone marrow, prostate, breast, colon, pancreas, stomach, esophagus, gastrointestinal tract, or reproductive tract (cervix, ovaries, endometrium etc.); and which produces shed tumor antigen (e.g., serous, or endometroid, or mucinous tumors). As apparent to one skilled in the art, lymphoid tumors, including B cell lymphomas, and leukemias are excluded from the definition of solid nonlymphoid tumors or their metastases. For the purposes of the present invention (including the specification and claims), “solid non-lymphoid tumor” may also include melanoma.

The term “tumor-associated antigen” is used herein, for purposes of the specification and claims, to mean a composition comprising one or more antigens expressed by tumor cells of solid nonlymphoid tumor origin. As apparent to one skilled in the art, as exemplified in Table 1 herein, tumor-associated antigen comprises a composition that may include whole tumor cells, hapten- or virus- or cytokine- modified tumor cells, a viral lysate of tumor cells, tumor cell lysate, tumor cell extract, tumor RNA (e.g., tumor RNA-pulsed dendritic cells), tumor-derived exosomes, a purified tumor antigen, a recombinantly produced tumor antigen, a synthetic tumor antigen (e.g., synthesized chemically), a combination of tumor antigens (polyvalent), tumor DNA (e.g., which when administered, produces one or more tumor antigens in cells which uptake and express the DNA), DNA encoding an anti-idiotype antibody which mimics an epitope of a tumor antigen (e.g., which when administered, produces a peptide or polypeptide which mimics tumor antigen in cells which uptake and express the DNA), one or more tumor antigens presented by antigen presenting cells, and a combination thereof. A tumor-associated antigen may be from autologous or allogeneic or semi-allogeneic (expresses both allogeneic and syngeneic determinants) tumor. A preferred tumor-associated antigen may be used in the present invention to the exclusion of tumor-associated antigen other than the preferred tumor-associated antigen. For example, in one preferred embodiment, an antibody comprising an anti-idiotypic antibody mimicking a tumor-associated antigen, may be excluded from tumor-associated antigen in the present invention (i.e., tumor-associated antigen is other than an anti-idiotypic antibody).

The term “shed tumor antigen” is used herein, for purposes of the specification and claims, to mean a glycomolecule (e.g., glycoprotein or glycolipid) which:

(a) by itself, or in an aggregated or oligomeric (two or more monomers which are together) form, has a molecular size equal to or greater than about 100 kilodaltons;

(b) is released (e.g., shed) from a primary solid nonlymphoid tumor or its metastases (“primary source”), thereby becoming soluble and allowing movement into lymphoid tissues regional or distal to the primary source;

(c) comprises a molecule which comprises a carbohydrate epitope present more than once on the molecule (e.g., the molecule has a plurality of carbohydrate chains, wherein several of the carbohydrate chains express the same carbohydrate epitope; hence the carbohydrate epitope is repeated in the structure of the molecule), wherein the carbohydrate epitope may include, but is not limited to, one or more of: Tn antigen (comprising a terminal N-acetyl galactosamine), or a terminal 2,6 linked sialic acid (e.g., sTn antigen comprising a terminal sialic acid 2,6-linked to N-acetyl galactosamine; or a terminal sialic acid 2,6-linked to galactose), the structures of which are known in the art;

(d) is capable of inducing a humoral immune response, which may ultimately result in the production and secretion of anti-shed tumor antigen antibody which is predominately of an IgG class; and

(e) can interact with anti-shed tumor antigen antibody in forming immune complexes, wherein the immune complexes may bind and crosslink Fc receptors (FcR) present on the surface of FcR-expressing cells.

For purposes of illustration, and not limitation, exemplifying such shed tumor antigen are mucins (e.g., the glycoprotein encoded by the MUC-1 gene) and mucin-like molecules (e.g., carcinoembryonic antigen (CEA), Sialyl Lewis a, and the like) produced and shed by solid, nonlymphoid tumor. For purposes of illustration, and not limitation, in a preferred embodiment of the present invention, the shed tumor antigen comprises the gene product of the MUC-1 gene (also known as polymorphic epithelial mucin). Shed tumor antigen and anti-shed tumor antigen antibodies may form immune complexes that may have a threshold level for spacing and number of antibody molecules necessary for Fc receptor (e.g., Fc gamma R) crosslinking.

The term “pro-tumor immune response”, for purposes of the specification and claims, means a humoral immune response against a terminal, carbohydrate epitope of shed tumor antigen resulting in the production of antibody (particularly IgG) to shed tumor antigen, wherein the antibody binds shed tumor antigen in forming immune complexes. Such immune complexes may promote tumor progression (one or more of tumor growth, invasion, or metastasis) by one or more mechanisms including, but not limited to, binding and crosslinking Fc receptors (FcR; e.g., Fc gamma R) on immune effector cells resulting in the release of inflammatory mediators which promote local tissue destruction and angiogenesis; and binding and crosslinking receptors expressed on endothelial cells resulting in an induction of endothelial cell proliferation and/or release of factors promoting angiogenesis. Immune effector cells are host cells which are mediators of inflammation and/or angiogenesis (e.g., one or more of granulocytes, macrophages, vascular endothelial cells) that are capable of inducing a cascade of processes which promote tumor progression. For example, after activation by such immune complexes, granulocytes and macrophages cooperate to release tissue degradative enzymes which breakdown the connective tissue matrix, thereby facilitating invasion of the tumor and spread of metastases beyond the primary tumor (see, e.g., Barbera-Guillem et al., Neoplasia 1:453-460, 1999).

The term “individual” is used herein, for purposes of the specification and claims, to mean a mammal, and preferably a human, who is at risk of developing, or has developed, a pro-tumor immune response. This may include an individual having one or more of: a primary tumor comprising a solid, nonlymphoid tumor and/or its metastases; a precancerous lesion comprising transformed (abnormal in proliferation and/or genetic makeup as compared to normal epithelial cells of the same type) cells of ductal epithelial origin which release shed tumor antigen; a high risk (e.g., environmentally and/or genetically, as recognized by those skilled in the art) for developing a solid nonlymphoid tumor; a risk of recurrence (e.g., an individual who has been treated for a solid nonlymphoid tumor and thereby inherently carries a risk of recurrence). The method and compositions according to the present invention are preferably intended for use to deplete nonmalignant B cells localized in lymphoid tissues and/or infiltrating a solid nonlymphoid tumor, and/or circulating in body fluids such as peripheral blood, and most preferably in individuals who have developed a pro-tumor immune response.

The term “vector” or “expression vector” is used herein for purposes of the specification and claims, to mean vectors used in accordance with the present invention as a vehicle for introducing into and expressing in a mammalian cell one or more desired genes. For example, the vector may comprise one or more genes, wherein the one or more genes may encode an anti-B cell agent, an immunomodulator, tumor-associated antigen, or a combination thereof. The one or more genes are expressed from the vector once the vector is introduced into a cell. As known to those skilled in the art, such vectors can be selected from plasmids, viruses, retroviruses, and the like. For a recent review of vectors useful in therapy of cancer, see Weichselbaum and Kufe (1997, Lancet, 349:S10-S12). The features of a vector which make it useful in the methods and compositions of the present invention include that it have a selection marker for identifying vector which has inserted therein the one or more genes (as described above); restriction sites to facilitate cloning of the one or more genes; the ability of the vector to enter and/or replicate in mammalian cells, and one or more control signals (promoter, enhancer, and the like) which facilitate expression of the one or more genes. Examples of a preferred vector for the in vivo introduction of a recombinant vector into mammalian cells include, but are not limited to viral vectors. Virus-based vectors are one preferred vehicle as they infect cells in vivo, wherein during the infection process the viral genetic material is transferred into the cells. A retroviral vector, such as a plasmid containing AAV (Adeno-associated virus) sequences, has been described previously (see for example Chatterjee et al., 1992, Science, 258:1485-1488; U.S. Pat. No. 5,252,479, herein incorporated by reference). In one embodiment, the AAV vector contains inverted terminal repeats (ITR) with a selection marker such as the gene encoding neomycin resistance, an SV40 promoter, a polylinker, and other plasmid sequences. A promoter in the ITR drives the expression of the neomycin phosphotransferase gene, whereas the SV40 promoter drives expression of the operably linked gene encoding an anti-B cell agent to be expressed. The inverted terminal repeats of the AAV vector provide a means for integrating the vector, and sequences inserted therein, into the chromosome as the repeats serve as a sequence which has been shown to insert site-specifically, rather than randomly, into chromosomes. Examples of other vectors for the in vitro or in vivo introduction into mammalian cells include, but are not limited to, retroviral vectors (Miller et al., 1989, BioTechniques 7:980-990; Korman et al., 1987, Proc. Nat/ Acad. Sci. USA 84:2150-54), papovavirus episomes (U.S. Pat. No. 5,624,820, herein incorporated by reference), and adenovirus vectors (U.S. Pat. No. 5,585,362, herein incorporated by reference). Such vectors can utilize tissue-specific promoters in targeting expression in cells in which expression is desired. For example, B cell-specific promoters are known to those skilled in the art to include, but are not limited to, immunoglobulin promoters (see, e.g., Thoger et al., 1997, Mol. Immunol 34:97-107; Luo and Roeder, 1995, Mol. Cell Biol. 15:4115-24; Cockerill and Klinken, 1990, Mol Cell. Biol. 10:1293-6), class II transactivator promoter (Lennon et al., 1997, Immunogenetics 45:266-73), mb-1 promoter (Fitzsimmons et al., 1996, Genes Dev. 10:2198-211; Travis et al., 1991, Mol. Cell. Biol. 11:5756-66), human B29 gene promoter (Thompson et al., 1996, Blood 87:666-73; Omori and Wall, 1993, Proc. Natl. Acad. Sci. USA 90:11723-7), and Fc epsilon RII promoter (Dierks et al., 1994, Mol. Immunol. 31:1181-89). As generally known to those skilled in the art, various promoters that may be used for expression in mammalian cells include, but are not limited to: human hemoglobin promoter, human muscle creatinine promoter, human actin promoter, human myosin promoter, Epstein Barr virus (EBV) promoter, cytomegalovirus (CMV) promoter, Moloney virus promoter, mouse mammary tumor virus (MMTV) promoter, human immunodeficiency virus long terminal repeat (HIV-LTR) promoter, and Rous sarcoma virus (RSV) LTR promoter. Likewise, a control element may additionally include an enhancer, and may be selected from enhancers of gene expression for the same genes listed as sources for promoter sequences. Additionally, various polyadenylation signals known to those in the art for directing expression in mammalian cells may include, but are not limited to: an SV40 polyadenylation signal, a beta-globin polyadenylation signals, a LTR polyadenylation signal, growth hormone polyadenylation signal, and a synthetic polyadenylation signal.

The development of vaccines to induce antitumor immunity is dependent on understanding tumor biology, including the immune responses induced by tumor as well as the interaction amongst the various types of host immune cells in an immune response induced by tumor. The present invention relates to: (a) the discovery of a humoral immune response, “a pro-tumor immune response”, which may be present in individuals bearing solid nonlymphoid tumor; and (b) that in an individual having a pro-tumor immune response, the pro-tumor immune response has a propensity (e.g., as mediated through activated B cells, immune complexes, and activated immune effector cells) to: selectively drive the immune response, in polarizing the immune response, to comprise a TH2 response; preserve an immune response polarized to a TH2 response; and to suppress cell mediated immune response comprising a TH1 response (as exemplified by a TH2/TH1 imbalance). The TH1 -suppressive nature of a pro-tumor immune response can render a cancer vaccine to be insufficient or ineffective in inducing a sustained TH1 response critical for antitumor immunity. Thus, a vaccine, which may otherwise be effective in inducing antitumor immunity in the absence of a pro-tumor immune response, is reduced in its ability to induce a sustained cell mediated immune response comprising a TH1 response in the presence of a pro-tumor immune response. The vaccines according to the present invention can be administered to reduce the TH2 response maintained by a pro-tumor immune response, to overcome local and/or systemic immunosuppression of a sustained cell mediated immune response, and to induce a sustained cell mediated immune response comprising a TH1 response in the development of antitumor immunity.

A pro-tumor immune response may contribute to a TH2/TH1 imbalance, and may contribute to suppression of a cell mediated immune response to tumor antigens, by one or more of the following mechanisms. First, shed tumor antigen is a soluble antigen, capable of inducing a strong humoral immune response. Thus, a tumor's chronic production of a soluble antigen, shed tumor antigen, appears to selectively induce development of antigen-specific TH2 cells, while inhibiting TH1 cell development. Additionally, shed tumor antigen may be presented by B cells, and B cells are efficient antigen presenting cells primarily for influencing T cells to differentiate into TH2 cells. More particularly, promotion of MHC-restricted B cell antigen presentation is associated with T cells secreting cytokines characteristic of a TH2 response. Secondly, B cells activated in a pro-tumor immune response, and immune effector cells activated in a pro-tumor immune response, produce one or more cytokines characteristic of a TH2 response. For example, a pro-tumor immune response may involve paracrine production of IL-10 by macrophages activated by immune complexes comprising shed tumor antigen and anti-shed tumor antigen antibody, and autocrine production of IL-10 by activated B cells. In another example, a pro-tumor immune response may involve paracrine production of IL-6 by macrophages activated by immune complexes comprising shed tumor antigen and anti-shed tumor antigen antibody, and autocrine production of IL-6 by activated B cells, in an amplification loop of B cell activation (resulting in an increasing number of B cells which become activated by shed tumor antigen). Also, tumor cells have been found to secrete IL-6, and also have IL-6 receptors. Thus, IL-6 produced in a pro-tumor immune response may act in a paracrine manner in inducing IL-6 production by tumor cells (similarly, IL-4 produced by a pro-tumor immune response may stimulate IL-10 secretion by tumor cells). Additionally, such cytokine production may further act to suppress a TH1 pattern (and also a cytotoxic T cell pattern) of cytokine production (e.g., suppression of IL-12 and/or IFN-γ production). In another example, a pro-tumor immune response may involve autocrine production of IL-4 by macrophages activated by immune complexes comprising shed tumor antigen and anti-shed tumor antigen antibody. As will be described herein in more detail, follicular dendritic cells present shed tumor antigen in lymphoid tissues which serve as a foci for a pro-tumor immune response. Further, cytokines present in the environment during antigen presentation may determine the type of immune response induced by follicular dendritic cells. Thus, in an additional mechanism by which a pro-tumor immune response may contribute to a Th2/TH1 imbalance, cytokines influence follicular dendritic cells to drive an immune response to TH2 response. For example, follicular dendritic cells that undergo maturation in the presence of IL-10 tend to have impaired capacity to induce a TH1 response, thereby favoring a TH2 response. In another example, antigen presentation by follicular dendritic cells, in the presence of IL-4 (e.g., such as produced by neighboring, immune complex-activated macrophages), favors development of a TH2 response. In another mechanism, immune complexes (comprised of shed tumor antigen and anti-shed tumor antigen antibody) continually formed in the process of a pro-tumor immune response may play an important role in the modulation of an immune response to shift to or to maintain a predominant TH2 response (e.g., such as by crosslinking Fc gamma receptors on Fc gamma receptor-expressing immune effector cells in activating the immune effector cells to produce cytokines characteristic of a TH2 response).

In some embodiments illustrated herein, it is important to consider the following concepts. Various strains of mice were used as a standard animal model for demonstrating a relationship between a pro-tumor immune response, a TH2/TH1 imbalance, and progression of solid nonlymphoid tumors. The present inventor has demonstrated that a pro-tumor immune response exists both in mice and in humans (e.g., as summarized in Example 1 herein) . More particularly, and in that regard, the same B lymphocyte pathology observed in humans in the progression of solid nonlymphoid tumor (see, e.g., Barbera-Guillem et al., 2000, Cancer Immunol. Immunother. 48:541-549) is also observed in mice bearing solid nonlymphoid tumor (of either murine or human origin); the presence of a pro-tumor immune response in vivo can be demonstrated by immunohistochemical analysis of sections of tumors obtained from mice and obtained from humans when analyzed for the presence of shed tumor-associated antigen and IgG antibody (i.e., in demonstrating that immune complexes comprised of shed tumor antigen can exist in tumor and regional lymphoid tissues); and the amount of immune complexes containing shed tumor antigen has been shown to be associated with tumor progression in both mice bearing solid nonlymphoid tumor and humans bearing solid nonlymphoid tumor. Additionally, it has been demonstrated that a TH2/TH1 imbalance is present in both mice bearing solid nonlymphoid tumor and humans bearing solid nonlymphoid tumors, and further that an immunomodulator can modulate the immune response of both tumor-bearing mice and tumor-bearing humans by inducing a transient TH1 response with a concomitant transient decrease in a TH2 response in shifting the TH2/TH1 imbalance (see, e.g., Sredni et al., 1996, J. Natl. Cancer Inst. 88:1276-1284). Similarly, in some of the following embodiments, in vitro cytokine release assays are used to demonstrate TH2 responses or cell mediated immune responses comprising a TH1 response (i.e., that may further include a cytotoxic CD8+ response). As apparent to one skilled in the art, such assays are generally accepted in the art for determining whether mononuclear cells (isolated from either humans bearing solid nonlymphoid tumor or mice bearing solid nonlymphoid tumor) are primed to respond in either a TH1 response and/or TH2 response, and that a cytokine pattern (e.g., either a TH1 pattern of cytokine production or a TH2 pattern of cytokine production) demonstrated from such assays is representative of the individual's immune response from whom the mononuclear cells were obtained.

In one embodiment, the present invention provides for a vaccine comprising an immunotherapeutic composition, and tumor-associated antigen. The vaccine may further comprise a component selected from an immunomodulator, a pharmaceutically acceptable carrier, and a combination thereof. The vaccine may be administered to an individual, whom has a TH2/TH1 imbalance caused by either a pro-tumor immune response or by a pro-tumor immune response and solid nonlymphoid tumor, in an amount effective to induce a cell mediated immune response comprising a TH1 response and to suppress (e.g., reduce) a TH2 response as relative to the immune response TH2/TH1 imbalance) of the individual before treatment. Reducing the TH2 response and inducing the TH1 response in the treated individual represents a “correction” (e.g., reduction) of a TH2/TH1 imbalance. Such correction of a TH2/TH1 imbalance may be demonstrated by determining a parameter comprised of the pattern of cytokine secretion in the individual (e.g., by assaying a clinical sample of body fluid (e.g., peripheral blood) for cytokine levels; or by obtaining mononuclear cells from the individual, and performing a cytokine release assay using the cells to determine the cytokine secretion pattern), the number of TH2 cells and/or TH1 cells in a clinical sample from an individual, or a combination thereof. For example, the parameter measured from the individual before treatment according to the method of the present invention is compared to the same parameter measured from the individual after treatment. Relative to this embodiment, provided is a method for immunotherapy of a TH2/TH1 imbalance in an individual, wherein the TH2/TH1 imbalance is effected by a disease process comprising a pro-tumor immune response, solid nonlymphoid tumor, or a combination thereof, wherein the method comprises administering to the individual a vaccine according to the present invention in an amount effective to induce a cell mediated immune response comprising a TH1 response and to suppress the TH2 response in the treated individual. Thus, provided is a method of reducing a TH2 response, and inducing a cell mediated immune response comprising a TH1 response against solid nonlymphoid tumor, in an individual having a TH2/TH1 imbalance, wherein the method comprises administering to the individual a vaccine according to the present invention in an amount effective to reduce a TH2 response and induce a cell mediated immune response comprising a TH1 response against solid nonlympholid tumor in the treated individual. Preferably the TH2 response that is reduced comprises a humoral immune response against shed tumor antigen (e.g., a humoral immune response as exemplified by a pro-tumor immune response), and the cell mediated immune response comprising a TH1 response induced is against tumor-associated antigen. In this preferred embodiment, the vaccine is for inducing (e.g., stimulating) a cell mediated immune response comprising a TH1 response against tumor-associated antigen (and hence against solid nonlymphoid tumor), and for reducing a TH2 response against shed tumor antigen (hence, allowing for developing and sustaining a cell mediated immune response comprising a TH1 response). The reduction of the TH2 response may be effected by depleting B cells in an individual by administering to the individual the immunotherapeutic composition of the vaccine in an amount effective to deplete B cells. The induction of a cell mediated immune response comprising a TH1 response may be effected by immunizing the individual with tumor-associated antigen of the vaccine. When the vaccine further comprises an immunomodulator, the immunomodulator is administered in an amount effective to induce a cell mediated immune response comprising a TH1 response. The induction of a cell mediated immune response comprising a TH1 response may be further facilitated (e.g., assisted in developing and/or sustaining the cell mediated immune response comprising a TH1 response) by the reduction of the TH2 response effected by the immunotherapeutic composition, as it is known in the art that a predominant TH2 response in an individual can suppress development of and/or maintenance of a cell mediated immune response comprising a TH1 response. Thus, the cell mediated immune response comprising a TH1 response induced by tumor-associated antigen of the vaccine may be further be effected by the immunomodulator, the immunotherapeutic composition, or a combination thereof. Likewise, in addition to reduction of the TH2 response effected by the immunotherapeutic composition, the reduction of the TH2 response may be further effected by inducing and sustaining a cell mediated immune response comprising a TH1 response effected by immunizing with tumor-associated antigen, by administering an immunomodulator, or a combination thereof. In that regard, it is known in the art that a predominant TH1 response may act to suppress the development of or reduce an existing TH2 response.

According to the present invention, provided is a vaccine and method for immunotherapy of an individual for treatment or prevention of solid nonlymphoid tumor in an individual. In one embodiment, the vaccine according to the present invention may be administered to an individual in an amount effective to prevent solid nonlymphoid tumor. For example, the individual may have a pro-tumor immune response but no detectable solid nonlymphoid tumor. Thus, the vaccine may be administered to the individual in an amount effective to inhibit growth of solid nonlymphoid tumor in the individual. In another example, the individual may have a pro-tumor immune response, and the individual's solid nonlymphoid tumor (e.g., primary tumor or primary tumor and any metastases) has been removed or reduced to a size that is not detectable (e.g., not detectable using current imaging methods such as magnetic resonance imaging, CAT scan, x-rays, ultrasound, or the like), by anticancer therapy (e.g., one or more of surgery, radiation therapy, photodynamic therapy, and the like). This individual is at risk for recurrence of tumor. Thus, the vaccine may be administered to the individual in an amount effective to inhibit growth (e.g., prevent recurrence) of solid nonlymphoid tumor in the treated individual. Relative to these embodiments, provided is a method for immunotherapy of an individual having a pro-tumor immune response, wherein the method comprises administering to the individual a vaccine according to the present invention in an amount effective to induce a cell mediated immune response comprising a TH1 response against tumor-associated antigen, and to suppress a TH2 response against shed tumor antigen, in the treated individual. As previously described herein in more detail, induction of the cell mediated immune response comprising a TH1 response may be effected by immunizing the individual with tumor-associated antigen, and may be further effected by administering an immunotherapeutic composition, an immunomodulator, or a combination thereof. As previously described herein in more detail, reduction of the TH2 response may be effected by administering an immunotherapeutic composition, and may be further effected by administering an immunomodulator, tumor-associated antigen (in immunizing the individual), or a combination thereof. With respect to use of the vaccine according to the present invention for immunotherapy of an individual for treatment of solid nonlymphoid tumor in the individual, administered to the individual is the vaccine according to the present invention. For example, the individual may have a pro-tumor immune response and solid nonlymphoid tumor. Thus, the vaccine may be administered to the individual in an amount effective to inhibit tumor progression of solid nonlymphoid tumor in the individual. As known in the art, and as defined herein, tumor progression comprises one or more of tumor growth, tumor invasion, metastasis. Preferably, to inhibit tumor progression comprises inducing a cell mediated immune response against the tumor, and reducing the TH2 response against shed tumor antigen, resulting in an antitumor effect. As previously described herein in more detail, induction of a cell mediated immune response comprising a TH1 response may be effected by immunizing the individual with tumor-associated antigen, and may be further effected by administering an immunotherapeutic composition, an immunomodulator, or a combination thereof. As previously described herein in more detail, reduction of the TH2 response may be effected by administering an immunotherapeutic composition, and may be further effected by administering an immunomodulator, tumor-associated antigen (in immunizing the individual), or a combination thereof. Preferably, in immunotherapy of an individual bearing a solid nonlymphoid tumor, the solid nonlymphoid tumor comprises a size of less than or equal to about 10 cm in diameter, and more preferably comprises a size of less than or equal to about 5 cm in diameter. Preferably, the solid nonlymphoid tumor comprises an early stage (stage 1 or stage 2) tumor. In that regard, and generally speaking, it is known in the art that current immunotherapy may have little or no anti-tumor effect against advanced tumors; i.e., most positive responses to immunotherapy to date have been obtained in patients with early stage tumors, suggesting (as would common sense) that immunotherapy should be reserved for patients with a relatively small tumor burden. Thus, the immunotherapy of the present invention is most effective when the tumor burden is small enough that it can be handled by the treated individual's immune system.

Also provided is a method of making a vaccine according to the present invention wherein the method comprises combining an immunotherapeutic composition, in an amount effective to deplete B cells, with tumor-associated antigen in an amount effective to induce a cell mediated immune response comprising a TH1 response. The method further comprises adding to the vaccine an immunomodulator, a pharmaceutically acceptable carrier, or a combination thereof. Further, provided is a tumor-associated antigen for use in a vaccine, and a method of making the tumor-associated antigen. The tumor-associated antigen according to the present invention comprises tumor antigens that have been formulated in micelles via their method of preparation. The tumor-associated antigen according to the present invention has advantages when compared to tumor-associated antigen comprising a purified component or whole tumor cells. For example, a micelle form of tumor-associated antigen (a) promotes cellular uptake of the tumor-associated antigen by antigen presenting cells (e.g., by an endocytosis mechanism), and (b) can behave like a macromolecular multivalent antigen.

The present invention is further illustrated by the following Examples.

### EXAMPLE 1

In this example, summarized and illustrated is that depletion of B cells can interrupt B cell involvement underlying a pro-tumor immune response in vivo, thereby inhibiting tumor progression. In one illustration of this example, fifty three C3H mice were injected intrasplenically with 10⁶ Met 129 tumor cells (high mucin-producing mammary carcinoma cells). The injected mice were then divided into two treatment groups. One group of 28 mice was injected with a control (not directed against any specific mouse antigen) goat IgG antibody (170 μg per injection) at days 5, 7, and 9 following tumor challenge. A second group consisted of 25 mice injected with goat anti-mouse IgG (170 μg per injection) at days 5, 7, and 9 following tumor challenge. The goat anti-mouse IgG was used to deplete the C3H mice of their B cells, thereby interrupting the host B cell-mediated pro-tumor immune response. At 22 days following tumor challenge, the two groups of mice were analyzed for primary tumor growth in the spleen (Table 2, “Tumor”), metastasis to the liver (Table 2, “Liver Met.”), and extra-regional metastasis (abdominal lymph nodes; Table 2, “Extra-R Met.”). Table 2 shows that there is a statistically significant reduction in the incidence of metastasis in the B cell-depleted mice (“Anti-IgG”) as compared to the control group receiving control IgG (“Goat-IgG Control”).

TABLE 2

| Observed | Goat-IgG Control | Anti-IgG |
|---|---|---|
| Tumor | 8 of 8 | 6 of 6 |
| Liver Met. | 5 of 8 | 0 of 6 |
| Extra-R Met. | 6 of 8 | 0 of 6 |

In summary, the results illustrated in Table 2 further support the finding that B cell depletion, such as depletion of shed antigen-specific B cells, can inhibit the in vivo pro-tumor immune response-mediated progression of solid nonlymphoid tumor.

Similar results have been observed in humans. More specifically, administered to several individuals having advanced cancer (Stage IV, solid nonlymphoid tumor) and a pro-tumor immune response was an immunotherapeutic composition comprising a chimeric anti-CD20 mAb in an amount effective to deplete B cells. To each individual was administered, by intravenous infusion, an initial dosage of 200 mg of the immuno-therapeutic composition; and then administered were at least two additional infusions, with each additional infusion spaced apart by four weeks from the previous infusion. The rate of infusion was dependent on how the individual tolerated infusion, the treating physician's judgment, drug manufacturer's instructions, and lack of side effects. At least two treated individuals showed a clinical benefit (e.g., reduction in the size and number of metastases) concomitant with a depletion of B cells (e.g., a reduction in shed antigen-specific B cells). However, it is not apparent that B cell depletion treatment, by itself, can induce and sustain a TH1 response to become dominant over the TH2 response present (e.g., as applied over a several month period, B cell depletion by itself does not appear to correct an existing TH2/TH1 imbalance).

### EXAMPLE 2

In this example, illustrated is a mechanism by which a pro-tumor immune response favors polarization of the immune response to a TH2 response in effecting a TH2/TH1 imbalance. As previously described herein in more detail, a pro-tumor immune response may contribute to a TH2/TH1 imbalance by one or more mechanisms. Relevant to this illustration, shed tumor antigen is a soluble antigen which is capable of inducing a strong humoral immune response resulting in the production of anti-shed tumor antigen antibody. Continuous and concomitant production of shed tumor antigen and anti-shed tumor antigen antibody results in immune complexes comprised of shed tumor antigen and anti-shed tumor antigen antibody. It has been discovered in the development of the present invention that these immune complexes play an important role in the modulation of an immune response to shift to &/or to maintain a predominant TH2 response (a TH2/TH1 imbalance). More particularly, these immune complexes can activate immune effector cells, such as macrophages, to produce a TH2 pattern of cytokines contributing to a TH2 response. To demonstrate this effect, an in vitro cytokine release assay was performed. Evaluated in the cytokine release assay were various combinations of components comprising murine macrophages (a murine macrophage cell line), human tumor cells (ductal breast carcinoma cell line T-47D) which are high secretors of shed tumor antigen comprising sTn-mucin (MUC-1), anti-shed tumor antigen antibody (anti-sTn mAb which is IgG), purified sTn antigen which is multivalent for sTn (bovine salivary mucin), and purified sTn antigen which is monovalent for sTn (sTn epitope). The components of each of the various combinations tested were mixed together in a well of a 24 well plate. In wells containing macrophages, 1.5×10⁵ cells/well were used; in wells containing tumor cells, 1.5×10⁴ cells/well were used; in wells containing anti-shed tumor antigen antibody, anti-sTn mAb was added to a final concentration of 0.06 μg/well; in wells containing purified multivalent sTn antigen, multivalent sTn antigen was added to a final concentration of 0.75 ng/well; and in wells containing purified monovalent sTn antigen, monovalent sTn antigen was added to a final concentration of 30 ng/well. The various combinations were incubated in cell culture medium for 24 hours at 37° C. in an incubator supplemented with 5% CO₂. Following the incubation period, the medium from each well was removed, centrifuged to remove any cells, and the resultant supernatants were collected for testing. Cytokines released by the macrophages were determined by an enzyme-linked immunosorbent assay specific for murine cytokines (commercially available ELISA kit, or commercially available service for performing the ELISAs) so as to distinguish the murine cytokines from any human cytokines that may be released by the tumor.

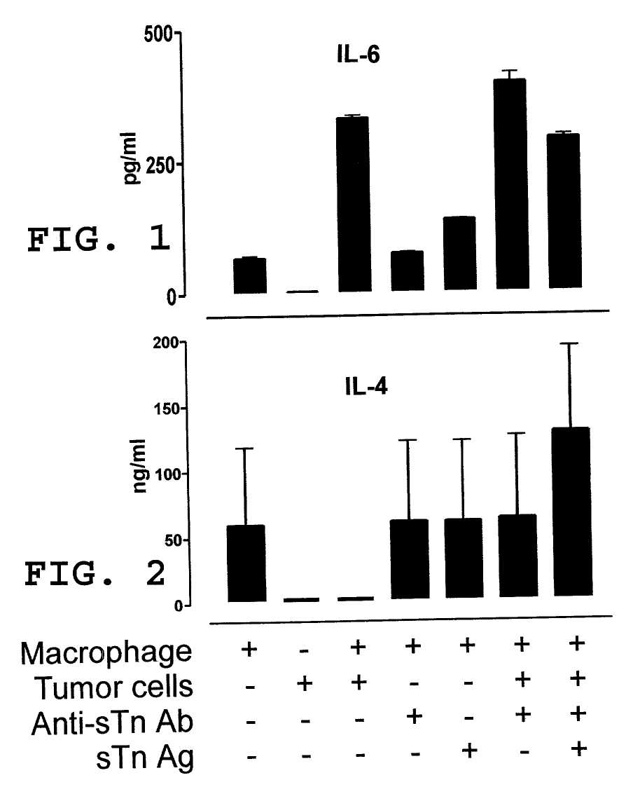

As illustrated in FIG. 1, macrophages alone produced an insignificant amount of the TH2 cytokine IL-6 (FIG. 1, first bar). In comparison, a significant amount of IL-6 was produced and secreted by macrophages when incubated with either tumor cells (FIG. 1, third bar), or tumor cells in the presence of anti-shed tumor antigen-antibody (anti-sTn mAb) (FIG. 1, sixth bar). The greatest induction occurred when the macrophages were incubated with both tumor cells and anti-shed tumor antigen-antibody. The fact that adding monovalent sTn antigen to this combination (FIG. 1, seventh bar) reduced the amount of IL-6 produced and secreted is an indication that immune complexes comprised of shed tumor antigen (produced and shed by the tumor cells) and anti-shed tumor antigen antibody are responsible for the significant increase in macrophage IL-6 production over that produced in the presence of tumor cells alone. More particularly, monovalent sTn antigen competes with shed tumor antigen (which is multivalent for sTn) in forming immune complexes, wherein an immune complex requires multivalent sTn for efficient crosslinking and activation of macrophages. Such a requirement for immune complex crosslinking has been demonstrated previously (Barbera-Guillem, 1999, supra). As illustrated in FIG. 2, and as compared to IL-4 produced by macrophages alone, a significant amount of TH2 cytokine IL-4 is produced only when macrophages are incubated with tumor cells, anti-shed tumor antigen-antibody, and multivalent sTn antigen (FIG. 2, seventh bar) . As illustrated in FIG. 3, induction of TH2 cytokine IL-10 production and secretion by macrophages occurred only when the macrophages were incubated with both the tumor cells and anti-shed tumor antigen-antibody (anti-sTn mAb) (FIG. 3, sixth bar). The fact that adding monovalent sTn antigen to this combination (FIG. 3, seventh bar) reduced the amount of IL-10 produced and secreted is an indication that immune complexes comprised of shed tumor antigen (produced and shed by the tumor cells) and anti-shed tumor antigen antibody are responsible for this significant increase in macrophage IL-10 production and secretion (for the reasons described above).

In summary, the data presented in FIGS. 1-3 confirm that macrophages, when activated by immune complexes such as produced in a pro-tumor immune response, can be activated to secrete a TH2 pattern of cytokines which contribute to a TH2 response and contribute to a TH2/TH1 imbalance in an individual having a pro-tumor immune response (e.g., in the presence of tumor, or even after removal of tumor).

### EXAMPLE 3

In this example, illustrated is a composition comprising micelles comprised of tumor-associated antigen for use in a vaccine, as well as a method of making the tumor-associated antigen. The tumor-associated antigen according to the present invention comprises tumor cell antigens that have been formulated in micelles via their method of preparation. Important features of the tumor-associated antigen according to the present invention is that it is substantially free of solubilizing agents (e.g., detergent-free and glycoside free) which are typically added to selectively solubilize components (e.g., addition of a detergent selectively solubilizes only certain components to the exclusion of other components not soluble in the detergent; glycosides selectively solubilize only charged monomeric proteins), further comprises a pharmaceutically acceptable carrier (i.e., a solution comprising a buffered solution, sterile water, or the like) is substantially free of oil (does not comprise oil added as an oil-in-water emulsion or oil adjuvant; as known to those skilled in the art, the use of oil in injections can result in unwanted side effects including, but not limited, abscesses, local granuloma formation, pyrogenicity, local pathological reactions, and the like), yet is capable of inducing a cell mediated immune response in an individual to whom the tumor-associated antigen is administered, particularly as a component of the vaccine according to the present invention. The amount of tumor-associated antigen useful in the vaccine according to the present invention may be determined empirically by standard experimentation well known by those skilled in the art without undue experimentation. Critical features of the tumor-associated antigen according to the present invention include that (a) tumor cells are lysed by a freeze-thaw process (which may better preserve the number, antigenicity, and structure of tumor antigens, with respect to its intended purpose, than lysis by sonication or detergents); (b) that the resultant tumor cell lysate is filtered to remove aggregates of cell components, any whole cells or large cell fragments (e.g., partially lysed cells); and (c) the filtered lysate is extruded through a filter of a pore size which induces micelle formation comprising tumor cell membrane fragments and other cellular components in the lysate (e.g., one or more of cytoplasmic components, intracellular proteins, mitochondria, nucleic acids, and the like). Micelle formation and quantitation may be confirmed by microscopy using standard methods known in the art. It is generally known in the art that there is a lack of cross-protection among individually derived tumors (see, e.g., Ramarathinam et al., 1995, J. Immunology 155:5323-5329). Thus, an additional feature of the composition comprising micelles comprised of tumor-associated antigen according to the present invention is the surprising discovery (unexpected result) that it can induce cross-protection against growth of solid nonlymphoid tumors of the same tissue but different clone (e.g., different tumor cell lines of the same tissue (e.g., colon carcinoma), or a tumor of the same tissue but from a different individual), against growth of solid nonlymphoid tumors of different tissues (tissue origin; e.g., colon carcinoma, lung carcinoma, and the like), or a combination thereof (induces cross-protection against both solid nonlymphoid tumors of the same tissue, and solid nonlymphoid tumors of different tissues). Thus, the composition, particularly when used in the vaccine according to the present invention, is capable of inducing an immunologic cross-protection against solid nonlymphoid tumors selected from the group consisting of solid nonlymphoid tumors of the same tissue but different origin than the solid nonlymphoid tumor from which the composition is produced, solid nonlymphoid tumors of different tissues than the solid nonlymphoid tumor from which the composition is produced, and a combination thereof. In that regard, mice immunized with the composition according to the present invention, comprising micelles comprised of tumor-associated antigen produced from lung carcinoma cells, cross-protected the mice against development of tumor when subsequently challenged by inoculation with either lung carcinoma cells or melanoma cells. A possible explanation is that the composition according to the present invention, particularly by its method of preparation, comprises at least one tumor antigen that is shared among tumor types, wherein induced is a cell mediated immune response comprising tumor-specific cytotoxic T cells that are capable of recognizing and lysing tumors derived from different tissues. As apparent to those skilled in the art, the tumor cells used to make the vaccine may be allogeneic, autologous, semi-allogeneic (see, U.S. Pat. No. 6,187,307), or a combination thereof (e.g., some cells may be allogenic, some cells may be autologous), with respect to the individual to receive a vaccine comprised of the tumor-associated antigen; additionally, the tumor cells may be tumor cells isolated from a tumor, an established tumor cell line, or a combination thereof (some cells may originate from one or more tumors, some cells may originate form one or more tumor cell lines).

A method for providing tumor-associated antigen for use in a vaccine comprises: (a) forming a pellet of tumor cells (e.g., such as by centrifugation or other means known in the art for pelleting cells); (b) exposing the pelleted tumor cells to a plurality of freeze/thaw cycles to disrupt the cells; (c) resuspending the disrupted cells, and any whole cells that may still be present, in a pharmaceutically acceptable carrier in forming a suspension; (d) filtering the suspension through a filter to remove any components greater than or equal to about 1 micron (e.g., whole cells, large cell fragments, nucleoli, and the like that may be present) in forming a filtered tumor cell lysate; and (e) extruding the filtered tumor cell lysate through a filter comprising pores of a size sufficient to induce formation of micelles in forming a composition comprising micelles comprised of tumor-associated antigen. This composition of tumor-associated antigen may be further processed in formulating the tumor-associated antigen for vaccine use (e.g., further dilution in a pharmaceutically acceptable carrier, and the like). In a preferred embodiment of forming a filtered tumor cell lysate, the suspension may be passed through a first filter (e.g., syringe filter or mesh for filtering) comprising pores of a size of greater than 1 micron but less than about 150 microns, and more preferably through pores of a size of about 100 microns, wherein the resultant filtrate (substantially free of large aggregates which could prevent (by clogging) or make difficult a second filtering) is then flowed through a second filter having a pore size of about 1 micron. The resultant filtered tumor lysate is then extruded through a filter comprising pores of a sufficient size to induce formation of micelles comprised of tumor-associated antigen. Preferably, a pore size sufficient to induce formation of micelles comprising the composition comprises a pore size in the range of from about 0.2 microns to about 0.7 microns, and more preferably comprises a pore size of about 0.5 microns. In a preferred embodiment, the micelles formed by extrusion comprise diameters that range from about 0.5 microns in diameter to diameters smaller than 0.5 microns. It is apparent to one skilled in the art that a freeze/thaw cycle comprises freezing the cells and then thawing the cells (e.g., until they are completely thawed). In a preferred embodiment, a plurality of freeze/thaw cycles comprises a number of cycles in the range of from about 2 to about 10.

For example, tumor-associated antigen was prepared for vaccine use by pelleting a suspension of 10⁶ tumor cells (in this example, Lewis lung carcinoma cell line- LLC1) in phosphate buffered saline (PBS) contained in a microfuge tube by using a microcentrifuge. The supernatant was removed from the pellet of tumor cells. The tube containing the tumor cells was placed in dry ice until the tumor cells were frozen (e.g., for a time ranging from about 3 minutes to about 10 minutes). The tube was then removed from the dry ice, and incubated at room temperature until the tumor cells were thawed (e.g., for a time ranging from about 5 minutes to about 30 minutes). The freeze/thaw cycle was repeated three times. The resultant tumor cell lysate was suspended in 50 μl of PBS and then filtered through a mesh of 100 micron pore size and then through a 1 micron pore size-syringe filter to remove whole cells and large cellular debris. The filtered tumor lysate was then extruded through a 0.5 micron pore size-syringe filter to facilitate formation of micelles comprising the tumor-associated antigen. The preparation of tumor-associated antigen was further diluted with PBS.

### EXAMPLE 4

In this example, illustrated is an embodiment for a vaccine according to the present invention. Also illustrated is an embodiment for a method of immunotherapy according to the present invention. As previously described herein in more detail, in one embodiment of the vaccine according to the present invention, the vaccine comprises an immunotherapeutic composition, and tumor-associated antigen. The vaccine may further comprise a component selected from the group consisting of an immunomodulator, a pharmaceutically acceptable carrier, and a combination thereof. While the invention is illustrated in this example with a form of tumor-associated antigen as described in more detail in Example 3 herein, it is apparent to those skilled in the art that other forms of tumor-associated antigen which are capable of inducing a cell mediated immune response comprising a TH1 response (see, Table 1) that is antitumor (e.g., against solid nonlymphoid tumor) may be useful in the vaccine and method accordance with the present invention. As apparent to one skilled in the art, antigens capable of inducing a cell mediated immune response typically include one or more T cell epitopes which, when presented to T cells by antigen presenting cells, results in clonal expansion of TH1 cells and/or cytotoxic T cells and/or expression of cytokines characteristic of a cell mediated immune response comprising a TH1 response. T cell epitopes (for inducing a TH1 response and/or a cytotoxic CD8+ T cell response) are recognized in the art as generally comprising linear peptide determinants that assume extended conformations within the peptide-binding cleft of MHC molecules. The linear peptides are defined by features involving charge, hydrophobicity, secondary structure (e.g., alpha-helical configuration), and amphipathic structure. Hence, segments of proteins which include T cell epitopes can be readily identified by identifying these features using computer programs well known in the art. A main objective during immunotherapy according to the present invention is to overcome the TH2/TH1 imbalance in the individual to be treated. As illustrated herein, this objective can be achieved by reducing the predominant TH2 response of a TH2/TH1 imbalance (mainly by administering an immuno-therapeutic composition) to facilitate the efficacy of a tumor-associated antigen, capable of inducing a cell mediated immune response, which is administered by itself (or with a pharmaceutically acceptable carrier) or in combination with an immunomodulator, a pharmaceutically acceptable carrier, or a combination thereof.

A standard animal model of tumor growth was used. More specifically, the vaccine according to the present invention was used in a method of immunotherapy of an individual at risk for recurrence of tumor. To illustrate this embodiment, mice (C57BL/6) received 10⁶ tumor cells (LLC1) subcutaneously. When the primary tumors in the mice reached 5 mm in diameter, the primary tumors were surgically removed. At this time, all animals had developed solid nonlymphoid tumor and a pro-tumor immune response. The mice were then divided into different groups. A first group of mice received no further treatment. The second, third, and fourth groups of mice were each treated with an immunotherapeutic composition so as to effect a B cell depletion. More particularly, on the day that the primary tumors were removed, mice of the second, third, and fourth groups were injected with a goat anti-mouse IgM antibody (each receiving 500 μg intraperitoneally) to effect a partial B cell depletion, as similar to the B cell depletion previously described in more detail in Example 1 herein. At day 1 after surgery (the day following surgery), mice in the third group received immunomodulator (IL-12, 30 ng, subcutaneously). At day 1 after surgery, mice in the fourth group received tumor-associated antigen (50 μl) mixed with an immunomodulator (IL-12, 30 ng) subcutaneously. At each of days 4 and 7 after surgery, mice in the third and fourth groups received a booster dose of immunomodulator (IL-12, 30 ng) in combination with immunotherapeutic composition (goat anti-mouse IgM antibody, 150 μg) administered intraperitoneally; whereas mice of the second group received booster doses of immunotherapeutic composition only.

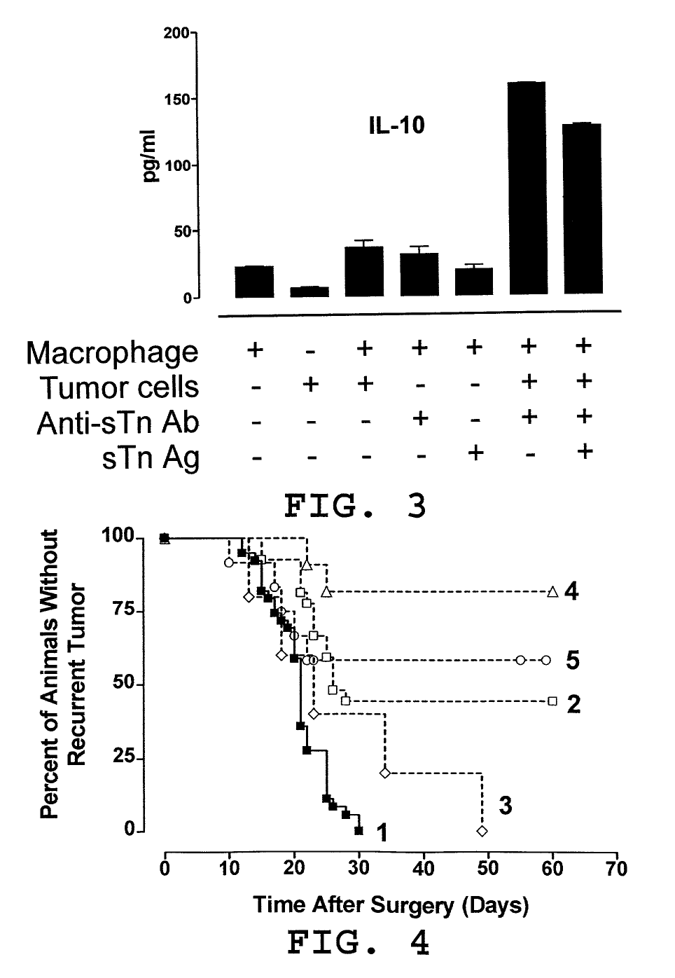

As shown in FIG. 4, all mice of group 1 (primary tumor resected and no other treatment) recurred with tumor (FIG. 4, line 1), and at least 20% of the mice had developed detectable metastases. Mice of group 3 (treated with immunotherapeutic composition and immunomodulator) developed recurrent tumor (FIG. 4, line 3) and metastases at a similar occurrence as that observed for mice in group 1. Less than half of the group of mice receiving immunotherapeutic composition alone were protected from recurrence (FIG. 4, line 2) and detectable metastases. In contrast, the majority (e.g., >60%) of mice of group 4, treated with a vaccine comprising an immunotherapeutic composition, tumor-associated antigen, and immunomodulator, failed to develop recurrence (FIG. 4, line 4) and also failed to develop detectable metastases.

In summary, the vaccine according to the present invention was effective in treating or preventing solid nonlymphoid tumor (e.g., in significantly inhibiting tumor progression such as tumor growth, metastasis) in individuals treated by a method of immunotherapy according to the present invention. It was observed that treatment with immunomodulator alone or tumor-associated antigen alone increased the rate of primary tumor growth. Thus, each of those treatments were excluded from the study illustrated in FIG. 4. However, the same effect can be seen in FIG. 4, where a treatment comprised of immunomodulator and immunotherapeutic composition (FIG. 4, line 3) resulted in a higher rate of recurrence and metastasis than a treatment with immuno-therapeutic composition alone (FIG. 4, line 2) . Accordingly, it is also demonstrated herein that, in the face of a pro-tumor immune, it may be insufficient to vaccinate with tumor-associated antigen by itself or in combination with immunomodulator and/or adjuvant. Rather, it may be necessary to control (reduce) the TH2 response as part of immunotherapy of an individual against solid nonlymphoid tumor.

To demonstrate that the vaccine according to the present invention is effective in correcting a TH2/TH1 imbalance in individuals having a TH2/TH1 imbalance, the pattern of cytokine secretion was compared between cells of different groups of mice treated as described in this Example 4. In that regard, spleen cells were analyzed for secretion of TH2 cytokine IL-4 and TH1 cytokine IFN-γ by ELISPOT assay according to the manufacturer's directions. The ratio of IL-4 to IFN-γ was used to express the TH2/TH1 imbalance. Mice of group 1 (primary tumor resected, and no other treatment) and mice of group 3 (treated with immunotherapeutic composition and immunomodulator) had an IL-4/IFN-γ ratio of about 0.75 as determined by ELISPOT. In contrast, mice of group 4 (treated with a vaccine comprising an immunotherapeutic composition, tumor-associated antigen, and immunomodulator) had an IL-4/IFN-γ ratio of only about 0.33 as determined by ELISPOT; and wherein the amount of IFN-γ secretion was significantly induced as compared to the pattern observed from mice of groups 1 & 3. Thus, the vaccine and method of immunotherapy was effective in correcting (reducing) the TH2/TH1 imbalance and pushing the immune response toward a dominant TH1 pattern of cytokine production. Another measurable indicator of a suppression of the TH2 response is the measurable reduction in titer of serum IgG (particularly IgG1) in mice treated with the vaccine according to the present invention.

### EXAMPLE 5

In this example, illustrated is embodiments for use of a vaccine according to the present invention. As described in more detail, the vaccine may be used in a method of immunotherapy of solid nonlymphoid tumor in an individual; and in a method for immunotherapy of a TH2/TH1 imbalance in an individual, wherein the TH2/TH1 imbalance is effected by a disease process comprising a pro-tumor immune response, solid nonlymphoid tumor, or a combination thereof. In one preferred embodiment, the vaccine comprises an immunotherapeutic composition, and tumor-associated antigen. In another preferred embodiment, the vaccine may further comprise a component selected from the group consisting of an immunomodulator, a pharmaceutically acceptable carrier, and a combination thereof. As described herein in more detail, the vaccine may be administered to an individual having a TH2/TH1 imbalance or, more preferably, a TH2/TH1 imbalance and a pro-tumor immune response. The individual may further have one or more of a pre-cancerous lesion, early stage cancer (Stage I or Stage II solid nonlymphoid tumors), metastases (e.g., wherein the primary tumor has been removed by surgery), or a high risk of recurrence. An objective of the vaccine is to administer the immunotherapeutic component of the vaccine in an effective amount to cause B cell depletion. B cell depletion may facilitate the correction of the TH2/TH1 imbalance in the individual.

In that regard, the immunotherapeutic composition of the vaccine may be administered to the individual to be treated at a time selected from the group consisting of before tumor-associated antigen of the vaccine is administered to the individual, simultaneous with the administration of tumor-associated antigen of the vaccine to the individual, subsequent to administration of tumor-associated antigen of the vaccine to the individual, or a combination thereof (where the immunotherapeutic composition is administered more than once to the individual; e.g., as illustrated in Example 3). In a preferred embodiment, the immunotherapeutic composition is administered to the individual before tumor-associated antigen is administered to the individual, and is also administered concomitantly with administration of tumor associated antigen to the individual. In a more preferred embodiment, the immunotherapeutic composition is administered to the individual before tumor-associated antigen is administered to the individual, is also administered concomitantly with administration of tumor associated antigen to the individual, and is further administered to the individual after tumor-associated antigen is administered to the individual; wherein the vaccine comprises multiple doses of immunotherapeutic composition. In this embodiment, the vaccine may further comprise multiple doses of tumor-associated antigen.

Similarly, the immunomodulator of the vaccine may be administered to the individual to be treated at a time selected from the group consisting of before tumor-associated antigen of the vaccine is administered to the individual, simultaneous with the administration of tumor-associated antigen of the vaccine to the individual, subsequent to administration of tumor-associated antigen of the vaccine to the individual, or a combination thereof (where the immunomodulator is administered more than once to the individual; e.g., as illustrated in Example 3). In a more preferred embodiment, the immunomodulator composition is administered concomitantly with administration of tumor associated antigen to the individual, and is further administered to the individual after tumor-associated antigen is administered to the individual; wherein the vaccine comprises multiple doses of immunotherapeutic composition and immunomodulator. In this embodiment, the vaccine may further comprise multiple doses of tumor-associated antigen.

In a preferred embodiment, the administration of the vaccine to an individual, in performing the method according to the present invention, is parenteral. The term “parenteral” includes administration intradermally, intravenously, intramuscularly, subcutaneously, rectally, vaginally, intraperitoneally, intratumorally, or a combination thereof (e.g., one component may be administered by one mode (e.g., intravenously), whereas the one or more remaining components may be administered by a different mode (e.g., subcutaneously)). As apparent to one skilled in the art, the mode(s) of administration will depend upon the composition of the various components of the vaccine. For example, the immunotherapeutic composition may preferably be administered intravenously, or by implanting a solid phase implant into the individual, wherein the solid phase implant contains the immunotherapeutic composition and provides a sustained delivery of the immuunotherapeutic composition to the inidividual (as will be described herein in more detail). Alternatively, wherein the individual bears solid nonlymphoid tumor, the immunotherapeutic composition may be administered in a site-directed manner (intratumorally) to the tumor tissue or organ containing the tumor by use of catheterization or functionally similar means to deliver the immunotherapeutic composition. Typically, tumor-associated antigen by itself, or in conjunction with immunomodulator, depending on the nature of each and as reviewed in the protocols for clinical trials of tumor-associated antigen by the National Cancer Institute, may be administered either subcutaneously, intradermally, or intratumorally (site-directed) in amounts and repeated dosages as recommended in the protocol for a specific clinical trial. As will be apparent to one skilled in the art, an amount of the vaccine effective for immunotherapy (“effective dosage”), and whether repeated dosages of the vaccine or any component thereof may be warranted, will depend on factors related to the individual to be treated which may include, but are not limited to: size, rate of metabolism, and overall health; overall immune status; severity of the pro-tumor immune response; severity of the TH2/TH1 imbalance; other treatments which the individual may be undergoing concurrently with the immunotherapy; mode(s) of administration of the vaccine; and pharmacokinetic and pharmacologic properties of the type of vaccine being used. As an illustrative example, an amount of immunotherapeutic composition as a vaccine component effective to deplete B cells in an individual may range from about 0.01 mg/kg of body weight to about 40 mg/kg of body weight per dose. However, as apparent to one skilled in the art, and in the discretion of a medical practitioner, a treatment may be warranted with a dosage falling inside or outside of this illustrative range. In an illustration of parenteral administration, an effective amount of the immunotherapeutic composition comprising a chimeric monoclonal antibody (e.g., chimeric anti-CD20 mAb or chimeric CD-22 mAb or chimeric CD21 mAb or a combination thereof) may be administered by intravenous injection (e.g., an initial dosage of an amount in a range of from about 200 mg to about 400 mg; and then administered may be at least two additional infusions, with each additional infusion spaced apart by four weeks from the previous infusion). In an illustrative alternative embodiment comprising a site-directed administration for colon cancer, an effective amount of such immunotherapeutic composition (e.g., in an amount in a range of from about 40 mg to about 200 mg) may be administered through a catheter via the celiac trunk, and a similar dose may be administered via the same catheter, through the superior mesenteric arteria. In either illustrative embodiment, the same or similar procedure may be repeated, depending upon changes in the immune status of the individual (e.g., a cell mediated immune response comprising a TH1 response; pro-tumor immune response; and effect on tumor progression), and measurable parameters of efficacy of the treatment. Various parameters may be used to monitor the effect of immunotherapy in the treated individual; wherein the parameters may include, but are not limited to, relative peripheral blood B lymphocyte counts (e.g., B cell phenotypes comprising CD19+ cells, CD19+ CD21 + cells, and/or CD19+ CD21+ sTn+ cells), the CD4/CD8 ratio of peripheral blood lymphocytes, the level and pattern of cytokine production (e.g., whether TH1 cytokines and/or TC1 cytokines are induced and/or TH2 cytokines are reduced), the number of TH2 cells and/or number of TH1 cells, serum concentration of shed tumor antigen and/or of IgG and IgM anti-shed tumor antigen antibody and/or immune complexes comprised thereof, blood tumor markers, and imaging of the solid nonlymphoid tumor (to assess tumor status in the individuals with such advanced cancer) after each treatment.

In another embodiment of use of a vaccine according to the present invention, provided is a method for immunotherapy of an individual for treatment or prevention of solid nonlymphoid tumor, wherein the method comprises administering to the individual a vaccine in an amount effective to reduce a TH2 response, and in an amount effective to induce a cell mediated immune response against solid nonlymphoid tumor; and wherein the vaccine comprises (a) an immunotherapeutic composition for effecting B cell depletion, and (b) tumor-associated antigen capable of inducing a cell mediated immune response comprising an immune response selected from the group consisting of a TH1 response, a cytotoxic CD8+ T cell response, and a combination thereof. In a preferred embodiment of this method, the vaccine is administered to the individual by administering a priming dose comprising the immunotherapeutic composition, and administering an immunizing dose comprising tumor-associated antigen. As will be described herein in more detail, the priming dose comprises one or more administrations of immunotherapeutic composition at a time prior to the immunizing dose (comprising one or more administrations of tumor-associated antigen), wherein the priming dose depletes B cells in reducing the TH2 response of the individual so as to facilitate the individual's immune system to respond with induction of a cell mediated immune response upon receiving the immunizing dose.

### EXAMPLE 6

In this example, illustrated is another embodiment for use of a vaccine according to the present invention, particularly when an individual receives a vaccine comprising multiple doses of immunotherapeutic composition, and wherein at least one dose of the multiple doses of immunotherapeutic composition is administered to the individual as a priming dose administered prior in time to (e.g., a time ranging from about 1 week to about 12 weeks before) administration of tumor-associated antigen to the individual. In this embodiment, it is an object of the invention to provide an effective method for priming the immune system of an individual to respond with an induction of a cell mediated immune response upon subsequent (to the initiation or completion of priming) administration of tumor-associated antigen to the individual (in one or more doses comprising an “immunizing” dose). Advantageously, the method provides a system for priming by administering a composition, such as an immunotherapeutic composition, to an individual so that the individual will produce a cell mediated immune response substantially immediately upon administration of an immunizing dose of tumor-associated antigen. As described herein in more detail, the priming dose comprises a composition selected from the group consisting of an immunotherapeutic composition, an anti-CD4 monoclonal antibody, or a combination thereof. The composition comprising the priming dose may further comprise a component selected from the group consisting of an immunomodulator, a pharmaceutically acceptable carrier, or a combination thereof. The one or more doses comprising the priming are administered in an effective amount to modulate the individual's immune system towards responding with induction of a cell mediated immune response, and more preferably toward a cytotoxic T cell response (as known to those skilled in the art as comprising cytotoxic CD8+ T cells sensitized to tumor-associated antigen), to tumor-associated antigen upon contact with the immunizing dose administered to the individual. The immunizing dose of tumor-associated antigen is administered in an amount effective to induce a cell mediated immune response in the individual. The immunizing dose may further comprise a component selected from the group consisting of an immunomodulator, a pharmaceutically acceptable carrier, or a combination thereof. Thus, methods of immunotherapy of an individual, as described in more detail in Example 5 herein, may comprise administering a priming dose and an immunizing dose to the individual.

In one preferred embodiment, the priming dose comprises multiple doses of the composition, wherein each dose of the multiple doses is administered separately (e.g., administration of a dose is spaced apart by a time period, such as a number of weeks, before the next successive dose of the multiple doses is administered). In another preferred embodiment, the priming dose comprises a single, extended sustained delivery (e.g., over a time period ranging from about 7 days to about 90 days) of the composition comprising the priming dose by administering to the individual a biocompatible and nontoxic solid phase implant containing the composition comprising the priming dose. In that regard, the implant provides extended sustained delivery of the composition comprising the priming dose into the surrounding tissue and body fluids (and more preferably, into the circulatory system) of the individual. In a preferred embodiment, the time period for sustained delivery of the composition comprising the priming dose comprises 7 to 90 bays before the immunizing dose is administered. In another preferred embodiment, the time period for sustained delivery of the composition comprising the priming dose comprises a time period ranging from 7 to 90 days before the immunizing dose is administered to 7 to 90 days since (after) the administration of the immunizing dose. Solid phase implants are known to those skilled in the art to comprise matrix materials that include, but are not limited to, sterols, derivatized sterols, cellulosic polymers, polylactide, polyamides, polycaprolactone, polyglycolide, polyesters, or other like polymers or copolymers thereof; and their methods of formulation and incorporation of compositions therein are well known in the art (see, e.g., U.S. Pat. Nos. 6,120,784, 5,939,380, 5,039,660, and 4,452,775, the contents of which are herein incorporated by reference). Preferably, the implant matrix material is biocompatible (e.g., causes no substantial tissue irritation or necrosis at site of implant), biodegradable (and/or bio-absorbable, e.g., after degraded, becomes absorbed by cells or one or more tissues of the individual) that, when implanted into the individual, will gradually disintegrate in the individual's system through chemical, enzymatic, metabolic, and/or cellular hydrolytic action (e.g., biodegradation), while releasing the composition comprising the priming dose contained therein. In addition to the composition comprising the priming dose, the implant may also contain (and deliver) a component selected from the group consisting of an immunomodulator, a pharmaceutically acceptable carrier, or a combination thereof. There are several advantages to use of an implant to deliver the priming dose. For example, the implant has advantages in producing less stress in an individual (e.g., does not require multiple visits by, and multiple injections of, the individual), a more consistent priming (wherein continuous amounts of the composition are received by the individual), and likely may require a lower overall amount of the composition, than a priming dose in which multiple doses of the composition are administered to the individual. The in vivo release rate and extent of release of the priming dose from the implant may be effectively controlled and optimized, for delivery of an effective amount of the composition comprising the priming dose and any additional component that may be present, by varying the formulation of the matrix material (e.g., with respect to size, shape, porosity, biodegradability, and the like) using methods known in the art. Administration of the implant is preferably performed by a qualified medical practitioner. Implantation is achieved by a suitable method known in the art to include, but is not limited to, surgical incision, catheterization, or use of a commercial injection gun. Administration to a site of the implant may comprise a suitable route including, but not limited to, subcutaneously, intramuscularly, intradermally, intratumorally, or the like. Once implanted, provided is sustained release from the implant of the composition into the surrounding tissues and circulatory system of the individual over the desired time period of priming, in an amount effective to modulate the immune system to respond by induction of a cell mediated immune response against tumor-associated antigen upon contact with an immunizing dose of tumor-associated antigen.

In one embodiment, the composition comprising the priming dose comprises an immunotherapeutic composition as previously described herein in more detail. An amount of such a composition, as a priming dose effective to modulate the immune system to respond in induction of a cell mediated immune response upon contact with an immunizing dose of tumor-associated antigen, comprises an amount of an immunotherapeutic composition effective for suppressing a TH2 response, as previously described herein in more detail. Suppression of a TH2 response in an individual having a TH2/TH1 imbalance will modulate the immune system of the individual to respond to an immunizing dose of tumor-associated antigen with induction of a cell mediated immune response, wherein the cell mediated immune response comprises an immune response selected from the group consisting of a TH1 response, a cytotoxic T cell response, and a combination thereof. The priming dose is easily determined by methods well known in the art, such as by conducting statistically valid immunization and challenge studies, as well as by monitoring indicators for a TH2 response as previously described herein in more detail. With the priming dose comprising a composition comprising an immunotherapeutic composition, preferably the immunotherapeutic composition comprises an affinity ligand having binding specificity for a determinant selected from the group consisting of CD19, CD20, CD21, CD22 (also known as LL2), CDIM, and Lym-1.

In another embodiment, the composition comprising the priming dose comprises an anti-CD4 monoclonal antibody in an amount effective to suppress a CD4+ T cell response in modulating the immune system of the individual to respond in induction of a cell mediated immune response comprising a cytotoxic CD8+ T cell response upon contact with an immunizing dose of tumor-associated antigen. In that regard, and as previously described herein in more detail, macrophages, dendritic cells, and B cells have a propensity to present soluble (shed) antigen to T cells in a MHC II-restricted manner. In development of the present invention, it has also been shown that CD4+ T cells (e.g., as TH2 cells) are capable of enhancing presentation of shed tumor antigen in potentiating a pro-tumor immune response (see, e.g., the disclosure of U.S. application No. 09/411,116). Additionally, as shown in FIG. 4, CD4+ T cell depleted mice show a significant reduction in both the rate of tumor recurrence (FIG. 4, line 5) and metastasis when compared to mice having an immunocompetent CD4+ immune system (FIG. 4, line 1). Thus, administering to an individual a composition comprising a priming dose in an amount effective to suppress a CD4+ T cell response, and administering to the individual an immunizing dose in an amount effective to induce a cell mediated immune response comprising a cytotoxic CD8+ T cell response, comprises immunotherapy of an individual in treatment or prevention of solid nonlymphoid tumor. It is known in the art that cytotoxic CD8+ T cells can be induced independent of CD4+ T cell help. Mechanisms proposed for induction of a CD8+ T cell response to antigen, in the absence of CD4+ T cells, include the expression of particular co-stimulatory molecules by antigen presenting cells to naïve T cells, and the presence of IFN-γ. Thus, in a preferred embodiment, the composition comprising the priming dose, or the immunizing dose, or a combination thereof, further comprises an immunomodulator which induces a TH1 pattern of cytokine secretion (e.g., preferably inducing IFN-γ production). A preferred immunomodulator, as previously described herein in more detail, comprises IL-12. Additionally, induction of a cytotoxic CD8+ T cell response (known in the art as a Tc1 response) may also contribute to correction of a TH2/TH1 imbalance in an individual, because cytotoxic CD8+ T cells (Tc1 cells) secrete a pattern of cytokines similar or substantially identical to the pattern of cytokines secreted by TH1 cells (e.g., IFN-γ & IL-2).

Anti-CD4 monoclonal antibodies have been used clinically for treatment of diseases such as rheumatoid arthritis, and Crohn's disease. Various anti-CD4 mono-clonal antibodies, including humanized or chimeric monoclonal antibodies, are well known in the art, and include, but are not limited to, M-T412, cM-T412, keliximab, 4162W94, PRIMATIZED™ anti-CD4 (IDEC-CE9.1), Centara™, BL4, and KT6. A priming dose may comprise one or more anti-CD4 monoclonal antibodies in an amount effective to suppress a CD4+ T cell response. It is generally known in the art that anti-CD4 monoclonal antibodies bind to cell surface CD4 and suppress a CD4+ T cell response by one or more mechanisms which include, but are not limited to, coating CD4+ T cells and causing a downmodulation of activity of the coated T cells, inhibiting CD4+ naive T cell activation (e.g., decreasing the sensitivity of the CD4+ T cells to antigen stimulation and/or antigen presentation), inhibiting CD4+ T cell proliferation, depleting CD4+ T cells, and causing a downmodulation of CD4+ T cells by prolonged occupation of cell surface CD4. In a preferred embodiment, the administration of the priming dose comprising anti-CD4 monoclonal antibody to an individual, in performing the method according to the present invention, is parenteral. As will be apparent to one skilled in the art, an amount of priming dose comprising anti-CD4 monoclonal antibody effective to suppress a CD4+ T cell response, in modulating the immune system of the individual to respond in induction of a cell mediated immune response comprising a cytotoxic CD8+ T cell response upon contact with an immunizing dose of tumor-associated antigen, will depend on factors related to the individual to be treated which may include, but are not limited to: size, rate of metabolism, and overall health; overall immune status; severity of the pro-tumor immune response; severity of the TH2/TH1 imbalance; other treatments which the individual may be undergoing concurrently with the priming; mode(s) of administration of the priming dose; as well as the pharmacokinetic and pharmacologic properties of the particular anti-CD4 monoclonal antibody comprising the priming dose administered. As an illustrative example, an amount of priming dose comprising anti-CD4 monoclonal antibody effective to suppress a CD4+ T cell response in an individual may range from about 0.01 mg/kg of body weight to about 40 mg/kg of body weight per dose; however, as apparent to one skilled in the art, and in the discretion of a medical practitioner, a treatment may be warranted with a dosage falling inside or outside of this illustrative range. For example, anti-CD4 monoclonal antibody has been administered: by a single intravenous infusion at a dose of between about 0.03 mg/kg of body weight to about 4 mg/kg of body weight; intravenously in five consecutive daily doses at a dose of between about 10 mg and about 300 mg; and by administering about 50 mg per day for 5 days, followed by a sustained continuous delivery of about 50 mg/week for 5 weeks.

Accordingly, in another embodiment of use of a vaccine according to the present invention, provided is a method for immunotherapy of an individual for treatment or prevention of solid nonlymphoid tumor, the method comprising administering to the individual a vaccine comprising: (a) a priming dose comprised of a composition selected from the group consisting of an immunotherapeutic composition, anti-CD4 monoclonal antibody, and a combination thereof; and (b) an immunizing dose comprised of tumor-associated antigen capable of inducing a cell mediated immune response comprising an immune response selected from the group consisting of a TH1 response, a cytotoxic CD8+ T cell response, and a combination thereof. As previously described herein in more detail, the vaccine may further comprise a component selected from the group consisting of an immunomodulator, a pharmaceutically acceptable carrier, and a combination thereof. In a preferred embodiment of this method, the priming dose may be administered as a solid phase implant containing the composition comprising the priming dose for delivery to the individual. With a vaccine wherein the priming dose comprises a composition comprising an immunotherapeutic composition, preferably the immunotherapeutic composition comprises an affinity ligand having binding specificity for a determinant selected from the group consisting of CD19, CD20, CD21, CD22 (also known as LL2), CDIM, and Lym-1. Also in a preferred embodiment, in this method the immunizing dose is administered to the individual at a time following administration of the primary dose to the individual (e.g., subsequent to the completion of administration of the priming dose to the individual, or subsequent to the initiation of the priming dose but before completion of the administration of the priming dose to the individual such as when the priming dose is administered both before and after administration of the immunizing dose). With a vaccine wherein the priming dose comprises a composition comprising anti-CD4 monoclonal antibody, preferably the immunizing dose induces a cell mediated immune response comprising a cytotoxic CD8+ T cell response.

### EXAMPLE 7

As illustrated in Examples 5 and 6, in some embodiments of the methods according to the present invention, the methods may comprise a regimen (e.g., course of immunotherapy) comprising administering a vaccine according to the present invention in dual doses (a “dual dose regimen”), wherein a first dose comprises administration of a priming dose, and a second dose comprises administration of an immunizing dose. Following a preferred dual dose regimen, the immunizing dose is administered at a desired time following administration of the priming dose (e.g., subsequent to the completion of administration of the priming dose, or subsequent to the initiation of the priming dose but before completion of administration of the priming dose, as previously described herein in more detail). As also previously described herein in more detail, the priming dose may further comprise a component selected from the group consisting of an immunomodulator, a pharmaceutically acceptable carrier, and a combination thereof; and the immunizing dose may further comprise a component selected from the group consisting of an immunomodulator, a pharmaceutically acceptable carrier, and a combination thereof.

Provided is a vaccination kit comprising the priming dose and immunizing dose, wherein the priming dose is contained in a separate container than the container containing the immunizing dose. The containers in the kit may contain the respective component (e.g., priming dose or immunizing dose) in single or multiple use vials, ampules, other suitable containers, or a container suitable for housing an implant as known in the art. The vaccination kit may further comprise instructional material (printed and/or computer-readable information) which may more fully describe the vaccine to be administered such as information which includes, but is not limited to, formulation or contents of the kit components, order and timing of administration of the kit components, as well as additional information of concern to a medical practitioner whom is to administer the vaccine. Thus, in one embodiment of the present invention, the vaccination kit comprises in separate containers: (a) a priming dose comprising a composition selected from the group consisting of an immunotherapeutic composition, anti-CD4 monoclonal antibody, and a combination thereof; and (b) an immunizing dose comprising tumor-associated antigen. In a preferred embodiment of the vaccination kit, the priming dose is contained in a sold phase implant for delivery of the composition comprising the priming dose over a desired period of time. The vaccination kit according to the present invention may further comprise a component selected from the group consisting of an immunomodulator, a pharmaceutically acceptable carrier, and a combination thereof; wherein the component may be contained in a separate container, or may be contained in the container containing the priming dose (e.g., formulated as part of the composition comprising the priming dose), or may be contained in the container containing the immunizing dose (e.g., formulated as part of the composition comprising the immunizing dose), or may be contained in both the container containing the priming dose and the container containing the immunizing dose. The vaccination kit may further comprise instructional material.

The foregoing description of the specific embodiments of the present invention have been described in detail for purposes of illustration. In view of the descriptions and illustrations, others skilled in the art can, by applying, current knowledge, readily modify and/or adapt the present invention for various applications without departing from the basic concept, and therefore such modifications and/or adaptations are intended to be within the meaning and scope of the appended claims.

What is claimed is:

1. A vaccine for suppressing a TH2 response and for inducing a cell mediated immune response comprising a TH1 response in an individual having a TH2/TH1 imbalance, the vaccine comprising: an immunotherapeutic composition for effecting B cell depletion; and tumor-associated antigen capable of inducing a cell mediated immune response comprising a TH1 response.

2. The vaccine according to claim 1, further comprising a component selected from the group consisting of an immunomodulator for inducing a cell mediated immune response comprising a TH1 response, a pharmaceutically acceptable carrier, and a combination thereof.

3. The vaccine according to claim 1, wherein the immunotherapeutic composition is contained in a solid phase implant for delivery of the immunotherapeutic composition.

4. The vaccine according to claim 1, wherein the immunotherapeutic composition further comprises an anti-B cell agent.

5. The vaccine according to claim 1, wherein the immunotherapeutic composition comprises an affinity ligand having binding specificity for a determinant selected from the group consisting of CD19, CD20, CD21, CD22 (also known as LL2), CDIM, and Lym-1.

6. The vaccine according to claim 1, wherein the immunotherapeutic composition comprises cobra venom factor.

7. The vaccine according to claim 1, wherein the TH2/TH1 imbalance is mediated by a disease process comprising a pro-tumor immune response.

8. The vaccine according to claim 1, wherein the TH2/TH1 imbalance is mediated by a disease process comprising a pro-tumor immune response and solid nonlymphoid tumor.

9. A vaccine useful for the treatment or prevention of solid nonlymphoid tumor in an individual, the vaccine comprising: an immunotherapeutic composition for effecting B cell depletion; and tumor-associated antigen capable of inducing a cell mediated immune response comprising a TH1 response.

10. The vaccine according to claim 9, further comprising a component selected from the group consisting of an immunomodulator for inducing a cell mediated immune response comprising a TH1 response, a pharmaceutically acceptable carrier, and a combination thereof.

11. The vaccine according to claim 9, wherein the immunotherapeutic composition further comprises an anti-B cell agent.

12. The vaccine according to claim 9, wherein the immunotherapeutic composition comprises an affinity ligand having binding specificity for a determinant selected from the group consisting of CD19, CD20, CD21, CD22 (also known as LL2), CDIM, and Lym-1.

13. The vaccine according to claim 9, wherein the immunotherapeutic composition comprises cobra venom factor.

14. A composition comprising micelles comprised of tumor-associated antigen, wherein the composition is substantially free of solubilizing agents, wherein the composition is substantially free of oil, wherein the tumor-associated antigen comprises tumor cells lysed by a freeze-thaw process, and wherein the composition further comprises a pharmaceutically acceptable carrier.

15. The composition according to claim 14, wherein the composition comprises micelles of tumor-associated antigen, wherein the micelles comprise diameters that range from about 0.5 microns in diameter to diameters smaller than 0.5 microns.

16. The composition according to claim 14, wherein the composition is capable of, inducing an immunologic cross-protection against solid nonlymphoid tumors selected from the group consisting of solid nonlymphoid tumors of the same tissue but different origin than the solid nonlymphoid tumor from which the composition is produced, solid nonlymphoid tumors of different tissues than the solid nonlymphoid tumor from which the composition is produced, and a combination thereof.

17. A method for immunotherapy of a TH2/TH1 imbalance in an individual comprising administering to the individual a vaccine in an amount effective to reduce a TH2 response, and in an amount effective to induce a cell mediated immune response comprising a TH1 response against solid nonlymphoid tumor, wherein the vaccine comprises:
an immunotherapeutic composition for effecting B cell depletion; and
tumor-associated antigen capable of inducing a cell mediated immune response comprising a TH1 response;
wherein the TH2/TH1 imbalance is mediated by a disease process selected from the group consisting of a pro-tumor immune response, solid nonlymphoid tumor, and a combination thereof.

18. The method according to claim 17, wherein the vaccine is administered to the individual by administering a priming dose comprising the immunotherapeutic composition, and administering an immunizing dose comprising tumor-associated antigen.

19. The method according to claim 17, wherein the vaccine further comprises a component selected from the group consisting of an immunomodulator for inducing a TH1 response, a pharmaceutically acceptable carrier, and a combination thereof.

20. The method according to claim 17, wherein the immunotherapeutic composition further comprises an anti-B cell agent.

21. The method according to claim 17, wherein the immunotherapeutic composition comprises an affinity ligand having binding specificity for a determinant selected from the group consisting of CD19, CD20, CD21, CD22 (also known as LL2), CDIM, and Lym-1.

22. The method according to claim 17, wherein the TH2 response reduced comprises a humoral immune response against shed tumor antigen.

23. The method according to claim 17, wherein the cell mediated immune response induced comprises a TH1 response against tumor-associated antigen.

24. The method according to 17, wherein the immunotherapeutic composition of the vaccine is administered to the individual at a time selected from the group consisting of before tumor-associated antigen of the vaccine is administered to the individual, simultaneous with the administration of tumor-associated antigen of the vaccine to the individual, subsequent to administration of tumor-associated antigen of the vaccine to the individual, and a combination thereof.

25. The method according to 19, wherein the vaccine further comprises an immunomodulator, and the immunomodulator is administered to the individual at a time selected from the group consisting of before tumor-associated antigen of the vaccine is administered to the individual, simultaneous with the administration of tumor-associated antigen of the vaccine to the individual, subsequent to administration of tumor-associated antigen of the vaccine to the individual, and a combination thereof.

26. The method according to 17, wherein the vaccine is administered parenterally.

27. A method for immunotherapy of an individual for treatment or prevention of solid nonlymphoid tumor, the method comprising administering to the individual a vaccine in an amount effective to reduce a TH2 response, and in an amount effective to induce a cell mediated immune response against solid nonlymphoid tumor, wherein the vaccine comprises:
an immunotherapeutic composition for effecting B cell depletion; and
tumor-associated antigen capable of inducing a cell mediated immune response comprising an immune response selected from the group consisting of a TH1 response, a cytotoxic CD8+ T cell response, and a combination thereof.

28. The method according to claim 27, wherein the vaccine is administered to the individual by administering a priming dose comprising the immunotherapeutic composition, and administering an immunizing dose comprising tumor-associated antigen.

29. The method according to claim 27, wherein the vaccine further comprises a component selected from the group consisting of an immunomodulator, a pharmaceutically acceptable carrier, and a combination thereof.

30. The method according to claim 27, wherein the immunotherapeutic composition further comprises an anti-B cell agent.

31. The method according to claim 27, wherein the immunotherapeutic composition comprises an affinity ligand having binding specificity for a determinant selected from the group consisting of CD19, CD20, CD21, CD22 (also known as LL2), CDIM, and Lym-1.

32. The method according to claim 27, wherein the TH2 response reduced comprises a humoral immune response against shed tumor antigen.

33. The method according to claim 27, wherein the cell mediated immune response induced comprises a cell mediated immune response against tumor-associated antigen.

34. The method according to 27, wherein the immunotherapeutic composition of the vaccine is administered to the individual at a time selected from the group consisting of before tumor-associated antigen of the vaccine is administered to the individual, simultaneous with the administration of tumor-associated antigen of the vaccine to the individual, subsequent to administration of tumor-associated antigen of the vaccine to the individual, and a combination thereof.

35. The method according to 28, wherein the vaccine further comprises an immunomodulator, and the immunomodulator is administered to the individual at a time selected from the group consisting of before tumor-associated antigen of the vaccine is administered to the individual, simultaneous with the administration of tumor-associated antigen of the vaccine to the individual, subsequent to administration of tumor-associated antigen of the vaccine to the individual, and a combination thereof.

36. The method according to 27, wherein the vaccine is administered parenterally.

37. A method for immunotherapy of an individual for treatment or prevention of solid nonlymphoid tumor, the method comprising administering to the individual a vaccine comprising:
a priming dose comprised of a composition selected from the group consisting of an immunotherapeutic composition, anti-CD4 monoclonal antibody, and a combination thereof; and
an immunizing dose comprised of tumor-associated antigen capable of inducing a cell mediated immune response comprising an immune response selected from the group consisting of a TH1 response, a cytotoxic CD8+ T cell response, and a combination thereof.

38. The method according to claim 37, wherein the vaccine further comprises a component selected from the group consisting of an immunomodulator, a pharmaceutically acceptable carrier, and a combination thereof.

39. The method according to claim 37, wherein the priming dose is administered as a solid phase implant containing the composition comprising the priming dose for delivery to the individual.

40. The method according to claim 37, wherein the priming dose comprises a composition comprising an immunotherapeutic composition, and the immunotherapeutic composition comprises an affinity ligand having binding specificity for a determinant selected from the group consisting of CD19, CD20, CD21, CD22 (also known as LL2), CDIM, and Lym-1.

41. The method according to claim 37, wherein the immunizing dose is administered at a time following administration of the primary dose to the individual.

42. The method according to claim 37, wherein the priming dose comprises a composition comprising anti-CD4 monoclonal antibody, and wherein the immunizing dose induces a cell mediated immune response comprising a cytotoxic CD8+ T cell response.

43. A method of making the vaccine according to claim 1, the method comprising combining an immunotherapeutic composition, in an amount effective to deplete B cells, with tumor-associated antigen, in an amount effective for inducing a cell mediated immune response comprising a TH1 response, in making the vaccine.

44. A method of making the vaccine according to claim 2, the method comprising combining an immunotherapeutic composition, in an amount effective to deplete B cells, with tumor-associated antigen, in an amount effective for inducing a cell mediated immune response comprising a TH1 response, with an immunomodulator in an amount effective for inducing a cell mediated immune response comprising a TH1 response, in making the vaccine.

45. A method of making the vaccine according to claim 2, the method comprising combining an immunotherapeutic composition, in an amount effective to deplete B cells, with tumor-associated antigen, in an amount effective for inducing a cell mediated immune response comprising a TH1 response, with a pharmaceutically acceptable carrier, in making the vaccine.

46. A method of making the vaccine according to claim 2, the method comprising combining an immunotherapeutic composition, in an amount effective to deplete B cells, with tumor-associated antigen, in an amount effective for inducing a cell mediated immune response comprising a TH1 response, with an immunomodulator in an amount effective for inducing a cell mediated immune response comprising a TH1 response, with a pharmaceutically acceptable carrier, in making the vaccine.

47. A method of making the vaccine according to claim 9, the method comprising combining an immunotherapeutic composition, in an amount effective to deplete B cells, with tumor-associated antigen, in an amount effective for inducing a cell mediated immune response comprising a TH1 response, in making the vaccine.

48. A method of making the vaccine according to claim 10, the method comprising combining an immunotherapeutic composition, in an amount effective to deplete B cells, with tumor-associated antigen, in an amount effective for inducing a cell mediated immune response comprising a TH1 response, with an immunomodulator in an amount effective for inducing a cell mediated immune response comprising a TH1 response, in making the vaccine.

49. A method of making the vaccine according to claim 10, the method comprising combining an immunotherapeutic composition, in an amount effective to deplete B cells, with tumor-associated antigen, in an amount effective for inducing a cell mediated immune response comprising a TH1 response, with a pharmaceutically acceptable carrier, in making the vaccine.

50. A method of making the vaccine according to claim 10, the method comprising combining an immunotherapeutic composition, in an amount effective to deplete B cells, with tumor-associated antigen, in an amount effective for inducing a cell mediated immune response comprising a TH1 response, with an immunomodulator in an amount effective for inducing a cell mediated immune response comprising a TH1 response, with a pharmaceutically acceptable carrier, in making the vaccine.

51. A method of making the composition according to 14, the method comprising:
(a) forming a pellet of tumor cells;
(b) exposing the pelleted tumor cells to a plurality of freeze/thaw cycles to disrupt the cells;
(c) resuspending the disrupted cells, and any whole cells that may still be present, in a pharmaceutically acceptable carrier in forming a suspension;
(d) filtering the suspension through a filter to remove any components greater than or equal to about 1 micron that may be present in forming a filtered tumor cell lysate; and
(e) extruding the filtered tumor cell lysate through a filter comprising pores of a size sufficient to induce formation of micelles in forming a composition comprising micelles comprised of tumor-associated antigen.

52. The method according to claim 51, wherein in forming a filtered tumor cell lysate, the suspension is passed through a first filter comprising pores of a size of greater than 1 micron but less than about 150 microns, and resultant filtrate is then flowed through a second filter comprising pores of a size of about 1 micron in forming a filtered tumor cell lysate.

53. The method according to claim 52, wherein the first filter comprises pores of a size of about 100 microns.

54. The method according to claim 51, wherein the filtered tumor lysate is extruded through a filter comprising pores of a size in the range of from about 0.2 microns to about 0.7 microns.

55. The method according to claim 51, wherein the filtered tumor lysate is extruded through a filter comprising pores of a size of about 0.5 microns.

56. The method according to claim 51, wherein the plurality of freeze/thaw cycles comprises a number of cycles in the range of from about 2 to about 10.

57. A method for priming the immune system of an individual, the method comprises:
administering to the individual a priming dose, wherein the priming dose comprises a composition selected from the group consisting of an immunotherapeutic composition, anti-CD4 monoclonal antibody, and a combination thereof;
wherein the priming dose is administered in an amount effective to modulate the individual's immune system to respond with induction of a cell mediated immune response upon administration of an immunizing dose of tumor-associated antigen to the individual.

58. The method according to claim 57, wherein the priming dose further comprises a component selected from the group consisting of an immunomodulator, a pharmaceutically acceptable carrier, and a combination thereof.

59. The method according to claim 57, wherein the priming dose is administered as a solid phase implant containing the composition comprising the priming dose for delivery to the individual.

60. The method according to claim 58, wherein the priming dose is administered as a solid phase implant.

61. The method according to claim 57, wherein the priming dose comprises a composition comprising an immunotherapeutic composition, and the immunotherapeutic composition comprises an affinity ligand having binding specificity for a determinant selected from the group consisting of CD19, CD20, CD21, CD22 (also known as LL2), CDIM, and Lym-1.

62. The method according to claim 57, wherein the priming dose comprises a composition comprising anti-CD4 monoclonal antibody, and wherein the priming dose modulates the individual's immune system to respond with induction of a cell mediated immune response comprising a cytotoxic CD8+ T cell response.

63. The method according to claim 58, wherein the priming dose comprises a composition comprising anti-CD4 monoclonal antibody and an immunomodulator, and wherein the priming dose modulates the individual's immune system to respond with induction of a cell mediated immune response comprising a cytotoxic CD8+ T cell response.

64. A vaccination kit comprising in separate containers:
(a) a priming dose comprising a composition selected from the group consisting of an immunotherapeutic composition, anti-CD4 monoclonal antibody, and a combination thereof; and
(b) an immunizing dose comprising tumor-associated antigen.

65. The vaccination kit according to claim 64, wherein the priming dose is contained in a sold phase implant for delivery of the composition comprising the priming dose over a desired period of time.

66. The vaccination kit according to claim 64, further comprising a component selected from the group consisting of an immunomodulator, a pharmaceutically acceptable carrier, and a combination thereof.

67. The vaccination kit according to claim 64, further comprising instructional material.

68. The vaccination kit according to claim 66, further comprising instructional material.
